(19) United States
(12) Patent Application Publication
(10) Pub. No.: US 2016/0058775 A1
(43) Pub. Date: Mar. 3, 2016

(54) COMPOSITION AND FORMULATION OF ANTIMICROBIAL AGENTS, PROCESSES THEREOF AND METHODS FOR TREATING MICROBIAL INFECTIONS

(71) Applicant: VYOME BIOSCIENCES PVT. LTD., New Delhi (IN)

(72) Inventors: Sudhanand PRASAD, New Delhi (IN); Sumana GHOSH, New Delhi (IN); Suresh Rameshlal CHAWRAI, New Delhi (IN); Nilu JAIN, New Delhi (IN)

(21) Appl. No.: 14/783,658

(22) PCT Filed: Apr. 12, 2014
(86) PCT No.: PCT/IB2014/060675

§ 371 Date: Oct. 9, 2015

(30) Foreign Application Priority Data
Apr. 12, 2013 (IN) 1103/DEL/2013

(51) Int. Cl. A61K 31/60; A61K 8/49; A61K 8/58; A61K 9/16; A61Q 5/06; A61Q 5/12; A61Q 5/02; A61K 8/02; A61K 31/496; A61Q 5/00

(52) U.S. Cl. CPC A61K 31/60; A61K 31/496; A61K 8/4926; A61K 8/58; A61Q 5/00; A61Q 5/06; A61Q 5/12; A61Q 5/02; A61K 8/0241; A61K 9/16

(57) ABSTRACT

The present disclosure provides compositions comprising antimicrobial agent and excipient, wherein the composition is devoid of fatty acids or their esters having more than 10 carbons. The present disclosure also provides compositions comprising antimicrobial agent and excipient, wherein the composition has at least one fatty acid/ester with carbon chain smaller than C11, and wherein the composition is devoid of fatty acids or their esters having more than 10 carbons. In an embodiment of the present disclosure, the compositions are a nanocomposite wherein particle size of at least one component is in nanoscale range. Further, the present disclosure also relates to formulating said compositions in a manner wherein, particle size or globule size of the formulation is in nanoscale range. The present disclosure also provides process for obtaining said compositions or formulations along with methods for treating microbial infections by using the compositions or the formulations of the present disclosure.

### TECHNICAL FIELD

The present disclosure provides compositions comprising antimicrobial agents and excipients, wherein the composition is devoid of fatty acids or their esters having more than 10 carbons. The present disclosure also provides compositions comprising antimicrobial agents and excipients, wherein the composition has at least one fatty acid/ester with carbon chain smaller than C11, and wherein the composition is devoid of fatty acids or their esters having more than 10 carbons. In an embodiment of the present disclosure, the compositions are a nanocomposite wherein particle size of at least one component is in nanoscale range. Further, the present disclosure also relates to formulating said compositions in a manner wherein, particle size or globule size of the formulation is in nanoscale range. The present disclosure also provides processes for obtaining said compositions or formulations along with methods for treating microbial infections by using the compositions or the formulations of the present disclosure.

### BACKGROUND AND PRIOR ART OF THE DISCLOSURE

Fungal infections of the skin are also known as ‘mycoses’. They are common and generally mild. In sick or otherwise immune-suppressed individuals, however, fungi can sometimes cause serious disease. Fungal infections in humans range from superficial, i.e., skin surface to deeply invasive type or disseminated infection.

In general, superficial fungal infections (also known as cutaneous mycosis) can affect the outer layers of skin, nails and hair. The main groups of fungi causing superficial fungal infections are dermatophytes (tinea), yeasts, e.g., candida, malassezia, piedra, etc. and moulds. These infections include dandruff/seborrheic dermatitis (D/SD), ringworm, onychomycosis, intertrigo, and those in psoriasis amongst others.

Seborrheic dermatitis is a common, chronic, superficial skin disorder causing scaly, itchy, red skin on the scalp, eyebrows, nasolabial creases, lips, ears, sternal area, axillae, submammary folds, umbilicus, groins, and gluteul crease. The disease is characterized by many shapes, sizes, and surface textures and is often crust-like, yellowish, and accompanied by itching. Seborrheic dermatitis is one of the leading causes of stubborn dandruff and occurs in all age groups. This condition primarily affects the sebaceous cysts present in the skin.

Currently, fungi of the genus Malassezia are believed to be the most likely responsible agents for causing dandruff (Dawson T. L., J. Investig. Dermatol. Symp. Proc. (2007), 12:1519). Most cases of seborrhoeic dermatitis likely involve an inflammatory reaction to the proliferation of the yeast Malassezia. These fungi are highly dependent on external lipids for in vitro growth (Chen T. A. and Hill P. V., Vet Dermatol, (2005), 16:4). Further, the inability to synthesize fatty acids may be complimented by the presence of multiple secreted lipases to aid in utilizing host lipids. Consequently, these fungi metabolize triglycerides present in sebum through these lipases resulting in lipid by-products.

The most common treatment of fungal infections is the topical application of antifungal agents that reduce the level of Malassezia on the scalp. Maintaining the scalp clean is mandatory for sufferers of seborrheic dermatitis. Use of effective anti-dandruff shampoos is therefore a significant way of preventing this condition.

Typically, the antifungal agent is applied to the scalp as a component of a shampoo or other hair care composition. The disadvantage of such shampoo formulations is that during normal usage the formulation does not remain on the scalp for a period of time sufficient to allow the antifungal agent to achieve its maximal therapeutic effect (Ralph M. Trüeb, JDDG, (2007), 5:356). These are designed to be applied, for example, in the shower or bath, and shortly thereafter rinsed off with water. Typically, the application instructions for such shampoos suggest that the formulation be removed after 3-5 minutes.

One of the antifungal agents, ketoconazole is among the most potent and widely used in anti-dandruff shampoos. However, the exposure time of shampoo is limited, due to which the efficacy is poor and relapse rates are higher.

In the past we found that, fatty acids and their derivatives (e.g. methylated and hydroxyl fatty acids) are known to possess antibacterial and antifungal activity as they target the cell membrane leading to increase in membrane fluidity (Douglas and Marshall and, “Antimicrobials in Food”, 3^{rd} edition, CRC Press 2005 Pg. no. 327-360).

In context to another review, the pelargonic and capric acid on Microsporum gypseum were found to be effective when tested in-vitro cell culture (Chandeganip our and Haims, “Mycoses”, 2001, Volume 44, Issue 3-4, pages 109-112). Similar reports were found with reference to Candida albicans when exposed to monoesters of glycerides of capric (C10 saturated medium chain fatty acid) (Bergsson et. al., Antimicrobial agents and Chemotherapy, 2001, Vol 45 pg. no. 3209-3212).

U.S. Patent Application 2010/0016271 discloses hair conditioning compositions comprising cationic surfactant, triglyceride oil and an anti-dandruff agent. These compositions contain triglyceride oil, which are fatty acid esters of glycerol, and hence act as nutrients and aid in the growth of the fungus. These compositions contain fatty material up to 10% having carbon chains from 8 to 30 carbon atoms.

U.S. Pat. No. 5,624,666 describes shampoo compositions containing anionic surfactants, cationic polymers, and zinc pyrithione as an anti-dandruff agent. It describes that conditioning agents such as silicone fluids can optionally be incorporated into the compositions therein. Head & Shoulders® Dandruff Shampoo Plus Conditioner is an example of a marketed product which provides both anti-dandruff and conditioning benefits upon application of the shampoo to hair. The exposure time of shampoos is less than required for effective antifungal activity, hence relapse rates are higher.

U.S. Pat. No. 7,547,752 refers to synergistic combination of an anti-dandruff agent with conjugated linoleic acid for prevention or treatment of dandruff and scalp itching.

European Patent No. 1923043A1 discloses cationic conditioning agents and an anti-dandruff agent with surfactants, siloxanes and natural and lipophillic oily components and their derivatives for the treatment or prevention of dandruff with conditioning.

European Patent No 0116439 discloses fatty acids like petroselinic and linoleic and saturated and unsaturated derivatives which alleviate dandruff and stimulate hair growth.

Commercially available formulations for the treatment of dandruff such as hair oils, styling gels, shampoos, etc, apart from having specific actives, usually also contain fungal fatty acid or their esters of carbon chains higher than C10 as essential ingredients. These fatty acid/esters in fact act as nutrients for fungi lacking fatty acid synthase (e.g., Malassezia sp.) and hence support their growth.

Accordingly, there remains a need for an antimicrobial composition comprising antibacterial, antiviral or antifungal agents that provides improved cleansing and optimal results, including anti-dandruff efficacy. The present disclosure addresses this need by providing topical compositions or formulations having antimicrobial agents and which is devoid of microbial nutrients.

### STATEMENT OF THE DISCLOSURE

Accordingly, the present disclosure relates to an anti-microbial composition comprising a) at least one anti-microbial agent, b) optionally at least one oil, or a fatty acid or ester thereof, or both and c) at least one excipient wherein said fatty acid or ester thereof is having less than 11 carbon atoms, wherein the composition is devoid of fatty acids or esters having more than 10 carbon atoms and wherein particle size of at least one component is in nano-scale range; a process for obtaining an anti-microbial composition as above, said process comprising act of: combining at least one anti-microbial agent with at least one excipient, optionally along with at least one oil, or a fatty acid or ester thereof, or both, in a manner such that at least one component has a particle size in nano-scale range and wherein the composition is devoid of fatty acids or esters having more than 10 carbon atoms; a method for treating a subject either suspected of having or having microbial infection, said method comprising act of administering to the subject an anti-microbial composition as above; anti-microbial composition as above, for use in treating microbial infection; and a kit for treating microbial infection, said kit comprising components selected from a group comprising antimicrobial agent, oil, fatty acid or ester thereof having less than 11 carbon atoms and excipient or any combination thereof along with an instruction manual.

### BRIEF DESCRIPTION OF THE ACCOMPANYING FIGURES

In order that the disclosure may be readily understood and put into practical effect, reference will now be made to exemplary embodiments as illustrated with reference to the accompanying figures. The figures together with a detailed description below, are incorporated in and form part of the specification, and serve to further illustrate the embodiments and explain various principles and advantages, in accordance with the present disclosure wherein:

FIG. 1: FIG. 1A shows globule size of ketoconazole emulsion gel (Composition A) analysed using Malvern Zetasizer. FIG. 1B shows transmission electron microscope image (IEM) of Composition A. FIG. 1C shows Scanning electron microscope image (SEM) of Composition A.

FIG. 2: shows representative bar diagrams of percentage of drug deposition in pig ear skin from marketed cream (non-nano) and Composition A for 3 hr and 6 hr residence time respectively on skin.

FIG. 3: shows a zone of inhibition (ZOI) of ketoconazole deposited in pig ear skin from marketed cream (non-nano) and Composition A after 3 hr and 6 hr residence time.

FIG. 4: FIG. 4A shows cream droplet size distribution of Composition B1 of hair cream formulation using ZetaSizer. FIG. 4B shows cream droplet size distribution of Composition B2 of hair cream formulation.

FIG. 5: FIG. 5A shows cream droplet size distribution of Composition C1 of hair gel formulation using ZetaSizer. FIG. 5B shows cream droplet size distribution of Composition C2 of hair gel formulation using DLS.

FIG. 6: shows size distribution data using ZetaSizer and Morphology & Particle Size by Scanning Electron Microscopy (SEM) and High-Resolution Transmission Electron Microscopy (HR-TEM) image of zinc pyrithione nanoparticles (Dispersion 1).

FIG. 7: shows size distribution data using ZetaSizer and Morphology & Particle Size by Scanning Electron Microscopy (SEM) and High-Resolution Transmission Electron Microscopy (HR-TEM) image of zinc pyrithione nanoparticles (Dispersion 2).

FIG. 8: shows dose Response Curves (Log Trend lines) of ZPT nanoparticles of the instant disclosure and micro particles (commercial ZPT powder), plotted using data of Zones of Inhibition on M. furfur.

FIG. 9: FIG. 9(A) shows time-kill of ZPT powder (10 μg/ml) with different concentrations of Capmul 908-P (0%, 3%, 5% & 9%) on M. furfur. FIG. 9(B): shows time-kill of ZPT powder (50 μg/ml) with different concentrations of Capmul 908-P (0%, 3%, 5% & 9%) on M. furfur.

FIG. 10: shows percentage of fungal inhibition at different time points after applying 10 mg of each formulation in 4 cm² freshly excised pig ear skin.

### DETAILED DESCRIPTION OF THE DISCLOSURE

While the disclosure is susceptible to various modifications and alternative forms, specific aspects thereof has been shown by way of examples and drawings and will be described in detail below. It should be understood, however, that it is not intended to limit the disclosure to the particular forms disclosed, but on the contrary, the intention is to cover all modifications, equivalents, and alternative falling within the spirit and the scope of the disclosure as defined by the appended claims.

In the present disclosure, reference is made to the examples mentioned to show only those specific details that are pertinent to understanding the aspects of the present disclosure so as not to obscure the disclosure with details that will be readily apparent to those of ordinary skill in the art having benefit of the description herein.

In the following detailed description of the aspects of the disclosure, reference is made to the accompanying drawings and graphs that form part hereof and in which are shown by way of illustration specific aspects in which the disclosure may be practiced. The aspects are described in sufficient details to enable those skilled in the art to practice the disclosure, and it is to be understood that other aspects may be utilized and that changes may be made without departing from the scope of the present disclosure.

The present disclosure relates to an anti-microbial composition comprising:
a) at least one anti-microbial agent;
b) optionally at least one oil, or a fatty acid or ester thereof, or both; and
c) at least one excipient;
   wherein said fatty acid or ester thereof is having less than 11 carbon atoms; wherein the composition is devoid of fatty acids or esters having more than 10 carbon atoms; and wherein particle size of at least one component is in nano-scale range.

In an embodiment of the present disclosure, the component having particle size in the nano-scale range is the anti-microbial agent.

In another embodiment of the present disclosure, the composition is formulated in a manner wherein, particle size or globule size of the formulation is in nanoscale range of about 1 nm to about 10,000 nm; preferably in the range of about 10 nm to about 1000 nm.

In yet another embodiment of the present disclosure, the formulation is a cream, oil, lotion, serum, gel, shampoo, nail varnish, ointment, foam, spray, conditioner, paste, mouthwash, sanitizer, solution, patch or aerosol.

In still another embodiment of the present disclosure, the anti-microbial agent is at a concentration ranging from about 0.01% to about 50% by weight of the total composition; preferably at a concentration ranging from about 0.01% to about 10% by weight of the total composition; and more preferably at a concentration ranging from about 0.01% to about 5% by weight of the total composition.

In still another embodiment of the present disclosure, the anti-microbial agent is selected from a group comprising anti-fungal agent, anti-bacterial agent and anti-viral agent or any combination thereof.

In still another embodiment of the present disclosure, the anti-fungal agent is selected from a group comprising piroctoneolamine, ciclopiroxolamine, ketoconazole, climbazole, miconazole nitrate, itraconazole, fluconazole, econazole, terconazole, saperconazole, amorolfine, oxiconazole, clotrimazole, luliconazole, terbinafine, butenafine, naftifine, selenium disulfide, salicylic acid, sulfur, tar, undecanoic acid, zinc pyrithione, hinokitol, arnica extract, walnut shell extract, tea tree oil, rosemary oil and birch oil or any combination thereof.

In still another embodiment of the present disclosure, the anti-bacterial agent is selected from a group comprising, macrolides, ketolides, beta lactams, monolactams, quinolones, sulfonamides, sulphathalidine, aminoglycosides, tetracyclines, rifamycins, glycopeptides, streptogramins, oxazolidinones, polymyxin, colistin, colymycin, trimethoprim, bacitracin, triclosan, besifloxacin, plurifloxacin, ornidazole, cephalothin, cefoxitin and phosphonomycin or any combination thereof.

In still another embodiment of the present disclosure, the anti-viral agent is selected from a group comprising Acylovir, Imiquimod, Docosanol, Penciclovir, Podophyllin, Podofilox, Aciclovir, Adefovir, Amantadine, Amprenavir, Arbidol, Atazanavir, Balavir, Boceprevirertet, Cidofovir, Combivir, Darunavir, Delavirdine, Didanosine, Edoxudine, Efavirenz, Emtricitabine, Enfuvirtide, Entecavir, Famciclovir, Fomivirsen, Fosamprenavir, Foscarnet, Fosfonet, Ganciclovir, Ibacitabine, Imunovir, Idoxuridine, Indinavir, Inosine, Integrase inhibitor, Lamivudine, Lopinavir, Loviride, Maraviroc, Moroxydine, Methisazone, Nelfinavir, Nevirapine, Nexavir, Nucleoside analogues, Oseltamivir, Peginterferon alfa-2a, Penciclovir, Peramivir, Pleconaril, Podophyllotoxin, Protease inhibitor, Raltegravir, Reverse transcriptase inhibitor, Ribavirin, Rimantadine, Ritonavir, Pyramidine, Saquinavir, Stavudine, Telaprevir, Tenofovir, Tenofovirdisoproxil, Tipranavir, Trifluridine, Trizivir, Tromantadine, Truvada, Valaciclovir, Valganciclovir, Vicriviroc, Vidarabine, Viramidine, Zalcitabine, Zanamivir, and Zidovudine or any combination thereof.

In still another embodiment of the present disclosure, the oil is either devoid of fatty acid or ester thereof or the oil comprises fatty acid or ester having less than 11 carbon atoms.

In still another embodiment of the present disclosure, the oil is selected from a group comprising paraffin oil, silicone oil, terpene, fatty alcohol, dibutyladipate, dioctyladipate, cetyl alcohol, stearyl alcohol and ceteryl alcohol or any combination thereof.

In still another embodiment of the present disclosure, the fatty acid or the ester thereof having less than 11 carbon atoms is selected from a group comprising propionic acid, butyric acid, pentanoic acid, hexanoic acid, heptanoic acid, caprylic acid, nonanoic acid, capric acid, mono or di ester of said acid with propylene glycol and mono or di or tri esters of said acid with glycerol, or any combination thereof; and wherein the fatty acid or the ester thereof is a part of the oil or an independent fatty acid or ester thereof.

In still another embodiment of the present disclosure, the oil or the fatty acid or the ester thereof is at a concentration ranging from about 0.5% to about 99% by weight of the total composition; preferably at a concentration ranging from about 50% to about 99% by weight of the total composition; more preferably at a concentration ranging from about 0.5% to about 20% by weight of the total composition.

In still another embodiment of the present disclosure, wherein the excipient is selected from a group comprising active agent, solvent, emulsifier, surfactant, polymer, stabilizer, oil and additive or any combination thereof.

In still another embodiment of the present disclosure, the active agent is selected from a group comprising pharmaceutical active, OTC active, anti-inflammatory agent and skin penetration enhancer or any combination thereof; solvent is selected from a group comprising C-1 to C-6 lower aliphatic alcohols, lower alkyl acetate, ethers, carboxylic acid, derivatives containing carbon chain length less than C11 and fatty alcohols or any combination thereof; wherein the emulsifier is selected from a group comprising steareth-2, Steareth-21, Poloxamer, Macrogolcetostearyl ether 20, cetyl alcohol cetearths, ceteth, isoceteths, laureths, oleths, steareths, lauramide DEA, and linoleamide DEA or any combination thereof; wherein the surfactant is selected from a group comprising Poloxamer, PEG-2 stearyl ether, PEG-21 stearyl ether, Pluoronic F127 (poloxamer), Polyoxyl 20 cetosteryl ether, Sodium laryl ether sulphate, Coco monoethanolamide, Cocamidopropylbetain, Sodium docusate and Ammonium lauryl sulphate or any combination thereof; and wherein the additive is selected from a group comprising, thickeners, antioxidants, perfumes or fragrances, essential oils, pH adjusters, herbal extracts, preserving agents, hair conditioning substances, hair care adjuncts, skin care adjuncts, emollient, dyestuffs, moisturizers, vitamins, sphingoceryls, sunscreens, co-surfactants, foaming agents, co-emulsifiers, viscosity modifiers, suspending agents, potentiating agents, pearlizing agents, cooling agents, ionic strength modifiers and oil-soluble polymers which are compatible with the base oil or skin care agents or both including skin-nutrient agents, anti-wrinkle agents, light and dust protectors or any combination thereof.

In still another embodiment of the present disclosure, the excipient is at a concentration ranging from about 0.5% to about 99.90% by weight of the total composition.

The present disclosure also relates to a process for obtaining an anti-microbial composition as above, said process comprising act of: combining at least one anti-microbial agent with at least one excipient, optionally along with at least one oil, or a fatty acid or ester thereof, or both, in a manner such that at least one component has a particle size in nano-scale range; and wherein the composition is devoid of fatty acids or esters having more than 10 carbon atoms.

In an embodiment of the present disclosure, the component is subjected to nanotization prior to the combining, or wherein the combination is subjected to homogenization to obtain the composition having the at least one component having a particle size in nano-scale range.

In another embodiment of the present disclosure, the homogenization of the combination results in in-situ generation of the nano-scale particles during the process for obtaining the composition.

In yet another embodiment of the present disclosure, the nanotization is carried out by a process comprising acts of:
a) combining the at least one component with a surfactant under stirring to obtain a suspension;
b) passing the resultant suspension through a homogenizer at high pressure and collecting the output dispersion; and
c) recycling the dispersion to obtain a nano dispersion having appropriately sized nanotized particles.

In still another embodiment of the present disclosure, the component having particle size in the nano-scale range is the anti-microbial agent.

The present disclosure also relates to a method for treating a subject either suspected of having or having microbial infection, said method comprising act of administering to the subject an anti-microbial composition as above.

In an embodiment of the present disclosure, the microbial infection is selected from a group comprising fungal infection, bacterial infection and viral infection or any combination thereof; and wherein the anti-microbial agent is selected from a group comprising anti-fungal agent, anti-bacterial agent and anti-viral agent or any combination thereof.

In another embodiment of the present disclosure, the fungal infection is caused by fungi selected from a group comprising Malassezia species, Trychophyton rubrum, Trychophyton mentagrophytes, Microsporum species, Epidermophyton species, Candida albicans and nondermatophyte molds or any combination thereof; wherein the bacterial infection is caused by bacteria selected from a group comprising Propionbacterium acnes, Staphylococcus species and Escherichia coli or any combination thereof; and wherein the viral infection is caused by virus selected from a group comprising herpes simplex virus, human cytomegalovirus, human adenovirus, hepatitis virus and human immunodeficiency virus or any combination thereof.

In yet another embodiment of the present disclosure, the subject is a mammal including a human.

In still another embodiment of the present disclosure, the administering of the composition is by a route selected from a group comprising oral, topical, dermal, mucosal, buccal and gum or any combination thereof.

The present disclosure also relates to anti-microbial composition as above, for use in treating microbial infection.

The present disclosure also relates to a kit for treating microbial infection, said kit comprising components selected from a group comprising antimicrobial agent, oil, fatty acid or ester thereof having less than 11 carbon atoms and excipient or any combination thereof along with an instruction manual.

The present disclosure is directed to compositions for the treatment of microbial infections, comprising:

(A) at least one antimicrobial agent; and
(B) at least one excipient,
wherein said composition is devoid of fatty acids/esters having carbon chain longer than C10.

The present disclosure is also directed to compositions for the treatment of microbial infections, comprising:

(A) at least one antimicrobial agent; and
(B) at least one excipient,
wherein said composition has at least one fatty acid/ester with carbon chain smaller than C11, and is devoid of fatty acids/esters having carbon chain longer than C10.

In an embodiment of the present disclosure, the composition is a nanocomposite wherein particle size of at least one component is in nanoscale range.

In another embodiment of the present disclosure, the component in the nano-scale range is the anti-microbial agent.

In yet another embodiment of the present disclosure, the composition of the present disclosure is formulated in a manner wherein, particle size or globule size of the formulation is in nanoscale range.

In yet another embodiment of the present disclosure, the term composition and formulation are used interchangeably.

The present disclosure is directed to compositions for the treatment of microbial infections, comprising:

(A) at least one antimicrobial agent; and
(B) at least one excipient,
wherein said composition is devoid of fatty acids/esters having carbon chain longer than C10; and wherein the composition is a nano-composite wherein particle size of at least one component is in nano-scale range, or wherein the composition is formulated in a manner wherein, particle size or globule size of the formulation is in nano-scale range.

The present disclosure is also directed to compositions for the treatment of microbial infections, comprising:

(A) at least one antimicrobial agent; and
(B) at least one excipient,
wherein said composition has at least one fatty acid/ester with carbon chain smaller than C11, and is devoid of fatty acids/esters having carbon chain longer than C10; and wherein the composition is a nanocomposite wherein particle size of at least one component is in nanoscale range, or wherein the composition is formulated in a manner wherein, particle size or globule size of the formulation is in nano-scale range.

The present disclosure also provides processes for obtaining said compositions or formulations along with methods for treating microbial infections by administering to a patient in need thereof, a composition or a formulation of the present disclosure.

In an embodiment of the present disclosure, the route for administering the composition to a patient is selected from a group comprising but not limiting to, oral, topical, dermal, mucosal, buccal and gum or any combination thereof.

In an embodiment of the present disclosure, the antimicrobial agent comprises an antifungal agent, antibacterial agent or antiviral agent, or any combination thereof. Further, the microbial infection may be a fungal infection, bacterial infection or viral infection, or any combination thereof.

In another embodiment of the present disclosure, the fungal infection is caused by a fungi selected from a group comprising Malassezia species, Trychophyton rubrum, Trychophyton mentagrophytes, Microsporum species, Epidermophyton species, Candida albicans and nondermatophyte molds or any combination thereof.

In yet another embodiment of the present disclosure, the bacterial infection is caused by bacteria selected from a group comprising Propionbacterium acnes, Staphylococcus species and Escherichia coli or any combination thereof.

In still another embodiment of the present disclosure, the viral infection is caused by a virus selected from a group comprising Herpes simplex virus, Human cytomegalovirus, Human adenovirus, Hepatitis virus and Human immunodeficiency virus or any combination thereof.

In still another embodiment of the present disclosure, the amount of antimicrobial agent used in the composition of the present disclosure is in the range of about 0.01% to about 50% by weight of the total composition. In yet another embodiment, the antimicrobial agent is in the range of from about 0.01% to about 10% by weight of the total composition. In a further embodiment, the antimicrobial agent is in the range of about 0.01% to about 5% by weight of the total composition.

In still another embodiment of the present disclosure, antifungal agent includes, but is not limited topiroctoneolamine, ciclopiroxolamine, ketoconazole, triclosan, climbazole, miconazole nitrate, itraconazole, fluconazole, econazole, terconazole, saperconazole, amorolfine, oxiconazole, clotrimazole, luliconazole, terbinafine, butenafine, naftifine, selenium disulfide, salicylic acid, sulfur, tar preparations, capric acid and derivatives, caprylic acid and derivatives, zinc pyrithione, hinokitol and chemical compounds from natural sources, such as extract of arnica, walnut shells, tea tree oil, rosemary oil, birch. Other antifungal agents known to the art-skilled may also be used in the compositions of the present disclosure.

In still another embodiment of the present disclosure, the antifungal agent used in the composition of the present disclosure is piroctoneolamine. In another embodiment of the present disclosure, the antifungal agent is ketoconazole. In yet another embodiment of the present disclosure, the antifungal agent is zinc pyrithione. In still another embodiment of the present disclosure, the composition comprises a combination of more than one antifungal agent.

In still another embodiment of the present disclosure, the term “antibacterial agent” is defined as a compound having either a bactericidal or bacteriostatic effect upon bacteria contacted by the compound. As used herein, the term “bactericidal” is defined to mean having a destructive killing action upon bacteria. As used herein, the term “bacteriostatic” is defined to mean having an inhibiting action upon the growth of bacteria.

In still another embodiment of the present disclosure, antibacterial agent includes, but is not limited to, macrolides or ketolides such as erythromycin, azithromycin, clarithromycin, and telithromycin; beta-lactams including penicillin, cephalosporin and carbapenems such as carbapenem, imipenem and meropenem; monolactams such as penicillin G, penicillin V, methicillin, oxacillin, cloxacillin, dicloxacillin, nafcillin, ampicillin, amoxicillin, carbenicillin, ticarcillin, meziocillin, piperacillin, azlocillin, temocillin, cepalothin, cephapirin, cephradine, cephaloridine, cefazolin, cefamandole, cefuroxime, cephalexin, cefprozil, cefaclor, loracarbef, cefoxitin, cefmetazole, cefotaxime, ceftizoxime, ceftriaxone, cefoperazone, ceftazidime, cefixime, cefpodoxime, ceftibuten, cefdinir, cefpirome, cefepime, and astreonam; quinolones such as nalidixic acid, oxolinic acid, norfloxacin, pefloxacin, enoxacin, ofloxacin, levofloxacin, ciprofloxacin, temafloxacin, lomefloxacin, fleroxacin, grepafloxacin, sparfloxacin, trovafloxacin, clinafloxacin, gatifloxacin, moxifloxacin, sitafloxacin, ganefloxacin, gemifloxacin and pazufloxacin; antibacterial sulfonamides and antibacterial sulphanilamides, including para-aminobenzoic acid, sulfadiazine, sulfisoxazole, sulfamethoxazole and sulfathalidine; aminoglycosides such as streptomycin, neomycin, kanamycin, paromycin, gentamicin, tobramycin, amikacin, netilmicin, spectinomycin, sisomicin, dibekalin and isepamicin; tetracyclines such as tetracycline, chlortetracycline, demeclocycline, minocycline, oxytetracycline, methacycline, doxycycline; rifamycins such as rifampicin (also called rifampin), rifapentine, rifabutin, bezoxazinorifamycin and rifaximin; lincosamides such as lincomycin and clindamycin; glycopeptides such as vancomycin and teicoplanin; streptogramins such as quinupristin and daflopristin; oxazolidinones such as linezolid; polymyxin, colistin and colymycin; trimethoprim, bacitracin, and phosphonomycin; Fluoro-quinolones such as Besifloxacin, Clinafloxacin, Garenoxacin Gemifloxacin, Moxifloxacin, Gatifloxacin, Sitafloxacin Trovafloxacin, Triclosan, Alatrofloxacin and Prulifloxacin.

In still another embodiment of the present disclosure, antiviral agent includes, but is not limited to, Acylovir, Imiquimod, Docosanol, Penciclovir, Podophyllin, Podofilox, Aciclovir, Adefovir, Amantadine, Amprenavir, Ampligen, Arbidol, Atazanavir, Atripla Balavir, Boceprevirertet, Cidofovir, Combivir, Darunavir, Delavirdine, Didanosine, Edoxudine, Efavirenz, Emtricitabine, Enfuvirtide, Entecavir, Famciclovir, Fomivirsen, Fosamprenavir, Foscarnet, Fosfonet, Ganciclovir, Ibacitabine, Imunovir, Idoxuridine, Indinavir, Inosine, Integrase inhibitor, Lamivudine, Lopinavir, Loviride, Maraviroc, Moroxydine, Methisazone, Nelfinavir, Nevirapine, Nexavir, Nucleoside analogues, Oseltamivir (Tamiflu), Peginterferon alfa-2a, Penciclovir, Peramivir, Pleconaril, Podophyllotoxin, Protease inhibitor (pharmacology), Raltegravir, Reverse transcriptase inhibitor, Ribavirin, Rimantadine, Ritonavir, Pyramidine, Saquinavir, Stavudine, Telaprevir, Tenofovir, Tenofovirdisoproxil, Tipranavir, Trifluridine, Trizivir, Tromantadine, Truvada, Valaciclovir, Valganciclovir, Vicriviroc, Vidarabine, Viramidine, Zalcitabine, Zanamivir (Relenza) and Zidovudine.

According to the present disclosure, excipient includes, but is not limited to, solvents, emulsifiers, surfactants, stabilizers, oils and additives used in pharmaceutical and cosmetic formulations. The amount of excipients used in the compositions of the present disclosure is in the range of about 0.5% to about 99.90% by weight of the total composition.

In an embodiment of the present disclosure, solvent includes, but is not limited to, C-1 to C-6 lower aliphatic alcohols, such as, for example, ethanol, isopropyl alcohol, butanol and the like, lower alkyl acetate, ethers, carboxylic acid and derivatives containing carbon chain length less than C11 (caprylic acid, capric acid and the like) or mixture/s thereof, and fatty alcohols such as undecanol, oleyl alcohol, lauryl alcohol or combinations thereof.

In another embodiment of the present disclosure, stabilizer includes, but is not limited to, surfactants, emulsifiers and polymers.

In yet another embodiment of the present disclosure, surfactant includes, but is not limited to, Poloxamer, PEG-2 stearyl ether, PEG-21 stearyl ether, Pluoronic F127 (poloxamer), Polyoxyl 20 cetosteryl ether, Sodium laryl ether sulphate, Coco monoethanolamide, Cocamidopropylbetain, sodium docusate and Ammonium lauryl sulphate,

In still another embodiment of the present disclosure, emulsifier includes, but is not limited to, Steareth-2, Steareth-21, Poloxamer, Macrogolcetostearyl ether 20 and cetyl alcohol.

In still another embodiment of the present disclosure, oil is either devoid of fatty acid or ester thereof or the oil comprises fatty acid or ester having less than 11 carbon atoms.

In still another embodiment of the present disclosure, oil includes, but is not limited to, paraffin oil, silicone oils, terpenes, fatty alcohols, dibutyladipate, dioctyladipate, cetyl alcohol, stearyl alcohol and ceteryl alcohol, or any combination thereof.

In still another embodiment of the present disclosure, less than C11 fatty acid and/or its ester includes, but is not limited to, propionic acid, butyric acid, pentanoic acid, hexanoic acid, heptanoic acid, caprylic acid, nonanoic acid, capric acid, mono/di ester of these acids with propylene glycol, mono/di/tri esters of these acids with glycerol, and combinations thereof.

In still another embodiment of the present disclosure, the amount of oil used in the compositions of the present disclosure is in the range of about 0.5% to about 99% by weight of the total composition. In another embodiment, the amount of oil used in the compositions of the present disclosure is in the range of about 50% to about 99% when formulated as oil, about 5% to about 50% when formulated as cream/ointment or about 0.5% to about 20% when formulated as gel/serum/spray.

In still another embodiment of the present disclosure, additives include, but are not limited to, thickeners, antioxidants, perfumes/fragrances, essential oils, pH adjusters, herbal extracts, preserving agents, hair conditioning substances, hair care adjuncts, skin care adjuncts, emollient, dyestuffs, moisturizers, vitamins, sphingoceryls, sunscreens, co-surfactants, foaming agents, co-emulsifiers, viscosity modifiers, suspending agents, potentiating agents, pearlizing agents, cooling agents, ionic strength modifiers and oil-soluble polymers which are compatible with the base oil and/or skin care agents including skin-nutrient agents, anti-wrinkle agents, light and dust protectors.

In still another embodiment of the present disclosure, essential oils include, but are not limited to, natural and synthetic oils such as eucalyptus oil, rosemary oil, pine needle oil, tea tree oil, sage oil, cinnamon oil, lemon oil, lime oil, orange oil, peppermint oil, spearmint oil, wintergreen oil, sweet birch oil, clove leaf oil, camphor oil, cardamon oil, cedar leaf oil, sweet birch oil and others known to the art-skilled.

In still another embodiment of the present disclosure, compositions of the present disclosure may contain additives such as thickeners (for example, bentonite, cellulose and the like), rheology modifiers (for example, carbopol, HPMC K100M, Cassia hydroxypropyltrimonium chloride and the like), polymers or fixing agent (for example, Polyvinylpyrollidone K90) antioxidants (for example, butylatedhydroxytoluene (BHT), butylatedhydroxyanisole (BHA), tert-butylhydroquinone (TBHQ), ferulic acid, Vitamin A, Vitamin E (Tocopherol)), preservatives (for example, methyl p-hydroxybenzoate or propyl p-hydroxybenzoate, di-sodium EDTA, Chloromethylisothiazolinone or Methylisothiazolinone, sorbic acid and the like), fragrances (for example, linalool), pearlizing agents, cooling agents (for example, menthol), ionic strength modifiers (for example, magnesium sulphate), hair care ingredients (for example, fatty alcohols, peptides, proteins, vitamins and mixtures thereof) and light protective agents or sunscreens (for example, p-methoxycinamic acid isoamyl ester and the like).

In still another embodiment of the present disclosure, surfactants include, but are not limited to, cetearths, ceteth, isoceteths, laureths, oleths, steareths, lauramide DEA, linoleamide DEA and other surfactants which are suitable for topical application.

In still another embodiment of the present disclosure, viscosity modifier may be used and includes, but is not limited to, polyethyleneglycol, propylene glycol, sodium chloride and Polyethylene glycol 600.

In still another embodiment of the present disclosure, foaming agent may be used and includes, but is not limited to, cocomonoethanolamide or others known to a person skilled in the art; suspending agent used herein includes cassia hydroxyl propyltrimonium chloride or others known to a person skilled in the art; potentiating agents used herein include zinc carbonate or others known to a person skilled in the art.

In still another embodiment of the present disclosure, pH adjusters include, but are not limited to, inorganic or organic acids (e.g., citric acid, lactic acid, succinic acid, acetic acid, fumaric acid, glycolic acid, benzoic acid), bases, salts and/or buffers thereof.

In still another embodiment of the present disclosure, herbal extracts include, but are not limited to, Amla fruit extract, Arnica Extract, Brahmi extract and others known to the art-skilled.

In still another embodiment of the present disclosure hair care adjuncts include, but are not limited to, ingredients beneficial in the treatment of hair loss or the promotion of hair growth such as taurine, caffeine, minoxidil, azelaic acid, marine cartilage, hydrolysed keratin, biotin, niacin, panthenol, vitamin B6, zinc, copper, peptides, horsetail silica, beta sitosterols, pycnogenol, PABA, green tea extract, folic acid, iron, L-cysteine, magnesium, ginseng and others known to the art-skilled.

In still another embodiment of the present disclosure conditioning agents include, but are not limited to, silicone fluid, stearamidopropyldimethylamine, cetrimonium chloride, polyquaternium-22, amodimethicone emulsion, cassia hydroxypropyltrimoniumchloride and others known to the art-skilled.

In still another embodiment of the present disclosure skin care adjuncts include, but are not limited to, those that are beneficial for the treatment of various skin conditions (like dry skin, oily skin, fine lines, pigmentation, etc.) such as proteins, vitamins (e.g., A, B, C, D, E, and K), trace metals (e.g., zinc, calcium and selenium), moisturizers (e.g., emollients, humectants, film formers, occlusive agents, and agents that affect the natural moisturization mechanisms of the skin), UV absorbers (physical and chemical absorbers such as para aminobenzoic acid (PABA), titanium dioxide, zinc oxide, etc.), anti-irritants (e.g., steroids and non-steroidal anti-inflammatories), botanical extracts (e.g., aloe vera, chamomile, cucumber extract, ginkgo biloba, ginseng, and rosemary), absorbents (e.g., aluminum starch octenylsuccinate, kaolin, corn starch, oat starch, cyclodextrin, talc, and zeolite), skin bleaching and lightening agents (e.g., hydroquinone and niacinamide lactate), humectants (e.g., glycerine, sorbitol, urea, Polyethylene glycol, Propylene glycol, Polyethylene glycol 600 and manitol), exfoliants, skin conditioning agents (e.g., aloe extracts, allanto in, bisabolol, ceramides, dimethicone, hyaluronic acid, and dipotassiumglycyrrhizate) and other natural components (e.g., oatmeal) known to the art-skilled.

In still another embodiment of the present disclosure, the composition or formulation is further supplemented with active agents. Such active agents include, but are not limited to pharmaceutical actives, OTC (over the counter) actives, anti-inflammatory agents and skin penetration enhancers. Further, skin penetration enhances include, but are not limited to TPGS (tocopheryl polyethylene glycol succinate), PEG (polyethylene glycol) or its esters. Such active agents in the present disclosure are categorized as excipient(s).

The present disclosure further provides methods for the treatment of microbial infections comprising administering to a patient in need thereof an antimicrobial composition of the present disclosure, said composition comprising at least one antimicrobial agent and at least one excipient, said composition being devoid of C11 or higher fatty acids and their esters.

In an embodiment of the present disclosure, the composition is a nanocomposite wherein particle size of at least one component is in nanoscale range.

The present disclosure further provides methods for the treatment of microbial infections comprising administering to a patient in need thereof an antimicrobial composition of the present disclosure, said composition comprising at least one antimicrobial agent and at least one excipient, said composition comprising at least one fatty acid/ester with carbon chain smaller than C11, and wherein said composition is devoid of C11 or higher fatty acids and their esters. In an embodiment of the present disclosure, the composition is a nanocomposite wherein particle size of at least one component is in nanoscale range.

The present disclosure also relates to formulating said composition in a manner wherein, particle size or globule size of the formulation is in nanoscale range. Such formulations may also be referred to as nanoformulations within the ambit of this disclosure.

In an embodiment of the present disclosure, the term “treatment” covers any topical microbial treatment in a mammal, such as a human.

In another embodiment of the present disclosure, the topical compositions or formulations thereof of the present disclosure are used in the treatment of diseases including, but not limited to, those associated with Malassezia, tineapedis, tineacapitis, tineacruris, tineaglabrosa, tineacorporis, onychomycosis, pityriasiscapitis, pityriasisvesicolor, pityrosporum folliculitis, seborrheicdermatitis. Further, the compositions or the formulations of the present disclosure are also used in the treatment of diseases associated with other fungi like Trychophyton rubrum or Trychophyton mentagrophytes or Microsporum species, or Epidermophyton species, or Candida albicans, etc. and other nondermatophyte molds. Furthermore, the compositions or formulations of the present disclosure are also of veterinary use in the topical treatment of dermatological fungal infections.

In yet another embodiment of the present disclosure, the compositions described herein can be used in personal care compositions, such as hair care compositions and skin care compositions.

For example, these personal care compositions can be used to treat or prevent dandruff. Compositions described herein can also be used in skin care compositions to treat or prevent, for example, acne. In some embodiments, the composition described herein can be used to treat a fungal or bacterial infection. For example, the composition described herein can be used to treat vaginal candidiasis, ring worm, (tinea infections of the body, scalp, beard, jock itch, and athlete's foot), nail infections, ear infections, and the like.

In another embodiment of the present disclosure, retinoids can be used with anti-microbial agent for treating acne.

In some embodiments of the present disclosure, retinoids are used in conjunction with anti-bacterial agents for curing acne.

In a yet another embodiment of the present disclosure, retinoids are selected from a group comprising Adapalene, Isotretinoin, Motretinide, Tazarotene and Tretinoin.

In a preferred embodiment of the present disclosure, the retinoid used is adapalene and is used in conjuction with besifloxacin of the present disclosure.

In still another embodiment of the present disclosure, the formulations provide better retention and penetration of antimicrobial agent onto the hair, skin, scalp and nails. Accordingly, the present disclosure provides formulations and methods of treating microbial infections of the skin, scalp, hair or nail.

The present disclosure relates to a process for obtaining said compositions or formulations of the present disclosure comprise acts of nanotizing at least one component to be used in the preparation of said compositions or formulations; or in situ generation of nanoscale particles/globules during the preparation of said compositions or formulations; or any other acts of preparation of said composition or formulations.

In still another embodiment of the present disclosure, resulting dispersion of nanoparticles is stabilized by addition of polymers including, but not limited to, carbopol Guar gum, xanthan gum, cassia gum or its derivatives, poly vinyl alcohol (PVA), polylactic-co-glycolic acid (PLGA) and polyethylene glycol (PEG), optionally followed by neutralization with a suitable base including but not limiting to, sodium hydroxide, potassium hydroxide, triethanolamine, di-isopropyl ethylamine and aminoethyl propanol.

The present disclosure further relates to formulations in various forms, such as, for example, oils, creams, lotions, serums, gels, ointments, foams, sprays, paste, mouthwash, sanitizer, solution or aerosols.

In an embodiment of the present disclosure, the antimicrobial composition of the present disclosure is formulated into nanoformulation of gel, wherein the gel is an emulsion gel. In another embodiment of the present disclosure, the antimicrobial composition is formulated into nano formulation of cream, wherein the cream is hair cream. In yet another embodiment of the present disclosure, the antimicrobial composition is formulated into nano formulation of gel, wherein the gel is hair gel. In still another embodiment of the present disclosure, the antimicrobial composition is formulated into nano formulation of shampoo. In still another embodiment of the present disclosure, the antimicrobial composition is formulated into nano formulation of conditioner.

Further, the present disclosure also relates to formulating said composition in a manner wherein, particle size or globule size of the formulation is in nano-scale range. Such formulations may also be referred to as nano formulations within the ambit of this disclosure.

In an embodiment of the present disclosure, the particle size or globule size of the composition being formulated is in nano-scale range, thereby providing for nano formulations of the present disclosure having size distribution or particle size or globule size in the range of about 1 nm to about 10,000 nm. In another embodiment, the range is about 10 nm to about 1000 nm.

Further, in the present disclosure, actives/agents (antimicrobial-antibacterial or antifungal or antiviral agents, etc.) in the form of either nanoparticle or nano-globule form show higher interaction with the stratum corneum (SC) of skin facilitating higher drug penetration through SC in comparison to their non-nano form. This fact overall enhances drug deposition in the epidermis, providing a reservoir of active agent in the epidermis that would translate higher fungal/bacterial/viral killing within short time period leading to improved therapeutic efficacy. The particle or globular size between 100-900 nm is considered to be optimum for very good retention in the skin epidermis.

The present disclosure relates to a method for treating or preventing dandruff or any skin related infections.

A more complete understanding can be obtained by reference to the following specific examples, which are provided for purposes of illustration only and are not intended to limit the scope of the disclosure. The examples form part of the detailed description of the instant invention.

### EXAMPLES

### Example 1

### Preparation of Ketoconazole Emulsion Gel Nano Formulation (Composition A)

In the present disclosure, composition A is in an emulsion gel form and in a nano-cream form.

Composition for preparation of ketoconazole emulsion gel is given as in Table 1 below:

TABLE 1

| | |
|---|---|
| Ingredient | Quantity (%) |

Phase A

| | |
|---|---|
| Ketoconazole | 2.0 |
| Lauryl alcohol | 3.3 |
| Propylene glycol monocaprylate | 3.3 |
| Steareth-2 | 3.3 |
| Steareth-21 | 3.3 |

Phase B

| | |
|---|---|
| Poloxamer | 6.5 |
| Glycerine | 3.0 |
| Water | q.s. |

Phase C

| | |
|---|---|
| Preservative | qs. |
| Fragrance | qs. |

Phase D

| | |
|---|---|
| pH Modifier | qs. |

Note:
qs = quantum satis

### Procedure for Preparation of Ketoconazole Emulsion Gelnano-Formulation

(1) Phase A: 20 mg Ketoconazole is solubilized in a mixture containing 33 mg Lauryl alcohol, 33 mg Sefsol 218, 33 mg Steareth 2 and 33 mg Steareth 21. Poloxamer (65 mg) is added into the mixture and the temperature is maintained at about 70-80° C.
(2) Phase B: Water phase contains 30 mg Glycerine and temperature is maintained at about 70° C. to about 80° C.
(3) Phase (A) is homogenized with phase (B) upon stirring (at about 700 rpm) and cooled to a temperature of about 35° C. to about 40° C. to obtain Phase C.
(4) The fragrance and preservative is added into Phase 3 while stirring at about 500 rpm, to obtain Phase D.
(5) 18% sodium hydroxide is added into Phase D to maintain pH ranging from about 5.0 to about 6.0.
(6) The pH, viscosity and globule size at different water dilution is measured.
In-situ nanotization occurs at the stage where Phase A is homogenized with Phase B.

### Measurement of the Globule Size

The globule size is measured by Malvern Zetasizer using dynamic light scattering (DLS) technique. It is further confirmed by Transmission electron microscope (TEM) and Scanning Electron Microscope (SEM) studies.

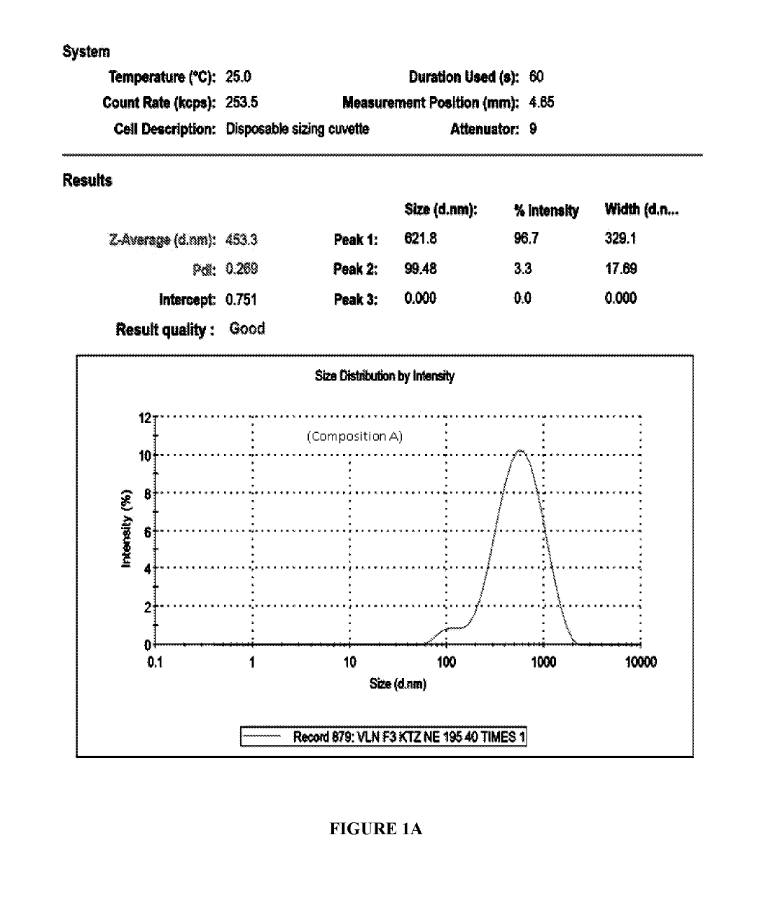

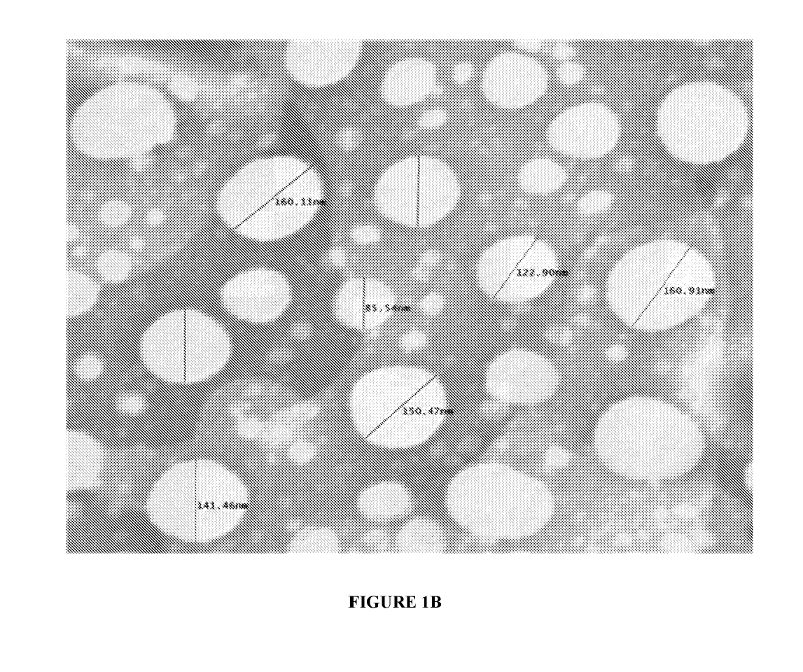

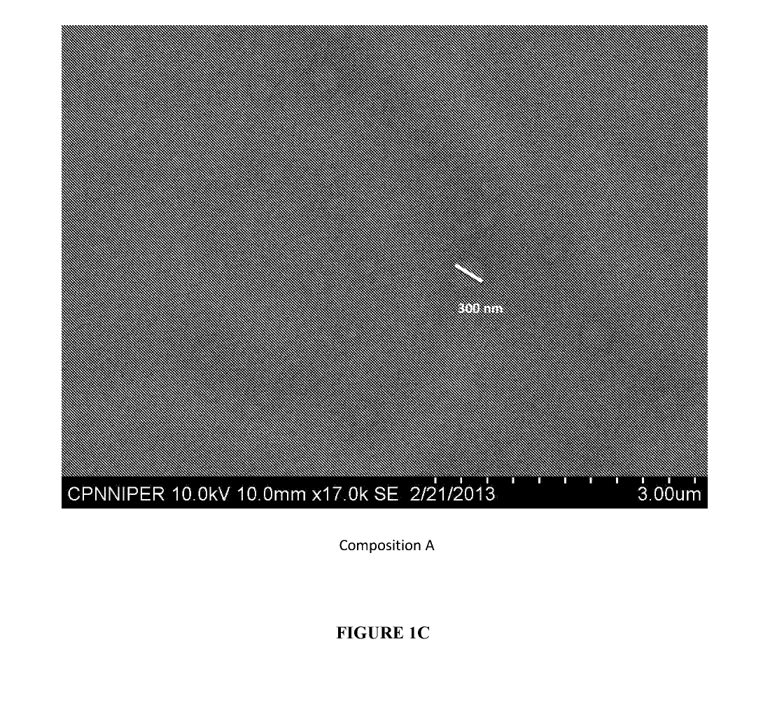

The size distribution of the Ketoconazole emulsion gel Nanoformulation (Composition A) is found to be in the range of about 100 nm to about 500 nm by different techniques. This confirms the nano distribution of oil droplets into the gel formulation. The results are summarized in following Table 2, which also compares the nanoformulation (Composition A) of the present disclosure with marketed non-nano formulation of ketoconazole, Nizoral. The Zetasizer, TEM and SEM pictures are shown in the FIGS. 1A, 1B and 1C respectively.

TABLE 2

| Name | Composition | Globule size | Viscosity |
|---|---|---|---|
| Non-nano | propylene glycol, stearyl | Zₐᵥ = 1420, | 4960 mPa · S |
| cream | alcohol, cetyl alcohols, | PDI = 0.311, | |
| | sorbitanmonostearate, | | |
| | polysorbate 60, isopropyl | | |
| | myristate, sodium sulfite | | |
| | anhydrous, polysorbate 80 | | |
| | and water | | |
| Instant | Lauryl alcohol, Caproyl 90, | Zav = 353.1, | 5288 mPa · S, |
| Composition | Steareth 2, Steareth 21, | PDI = 0.585, | |
| A emugel | Poloxamer 407, Propylene | | |
| | glycol, water, preservative, | | |

### Example 2

### Retention Studies of Ketoconazole Emulsion Gel Nano Formulation (Composition A) on Ex-Vivo Pit Skin Model and Comparison with Marketed Ketoconazole (Nizoral) (Non-Nano) Formulation

This example compares in-vitro skin penetration rate and distribution of marketed 2% ketoconazole cream against the 2% ketoconazole emulsion gel Nanoformulation (Composition A) of the present disclosure mentioned in Example 1. The experiment is performed using fresh pig skin mounted on Franz cell assembly. The receptor chamber is filled with phosphate buffered saline (PBS, pH 7.4), and the skin surface is mounted on top of the assembly. The skin is equilibrated at temperature about 32±1° C. for 1 h. The formulations are applied to the skin surface at a dose of about 0.815 mg/cm² and cap of the franz cell is properly clamped on top of this. Three replicates are run for each formulation.

After 3 hrs and 6 hrs, the diffusion cells are dismantled, washed with about 50 ml PBS buffer (pH about 7.4) followed by four times wiping with cotton bud to remove the cream present on skin surface. The drug deposited onto about 4.9 cm² area of the skin surface is extracted by about 10 ml methanol using homogenization for about 5 min followed by sonication for about 10 min. Finally the sample is centrifuged and supernatant is filtered and an aliquot of each sample is analyzed by HPLC to obtain ketoconazole content on each skin surface. Ketoconazole amount present in the full-length skin section is determined (see Table 3).

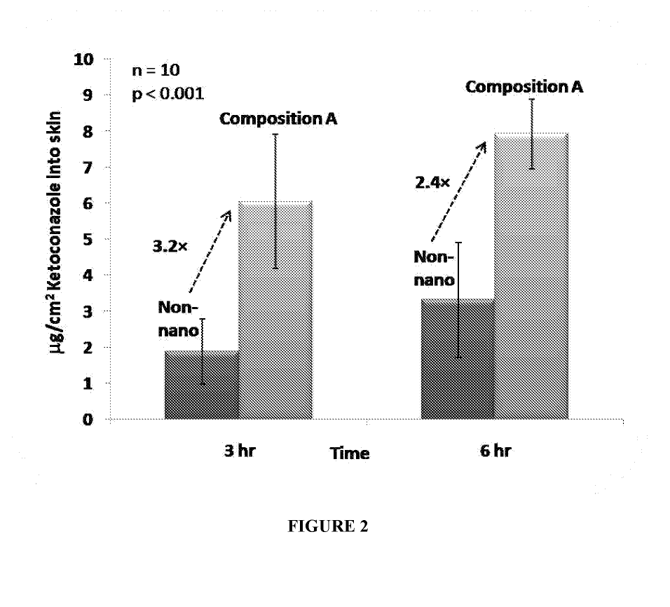

The results of ketoconazole deposition in pig skin (in-vitro) from marketed cream (non-nano) and formulation (Composition A) of the present disclosure (3 hour and 6 hour residence time) is summarized in Table 3 and depicted in FIG. 2.

TABLE 3

| | Average amount | Average drug | Average drug |
|---|---|---|---|
| | of drug | deposition into | deposition into |
| | (Ketoconazole) | skin after 3 hr | skin after 6 hr |
| | applied on | application (μg/ | application (μg/ |
| Sample name | the skin | cm² of skin) | cm² of skin) |
| Non-nano cream | 1.3 mg | 1.88 | 3.31 |
| (2% Ketoconazole) | | | |
| Instant formulation | 1.3 mg | 6.045 | 7.92 |
| (Composition A, | | | |
| 2% Ketoconazole) | | | |

The results provided herein, demonstrate higher (3-4 times) skin penetration properties of nanoformulation of ketoconazole emulsion gel (Composition A) in comparison to marketed non-nano formulation under similar experimental condition. The higher retention thus leads to better efficacy in curing fungal infection, which is demonstrated in Example 3 below.

### Example 3

### Comparative In Vitro Bio-Efficacy Studies of Ketoconazole Emulsion Gel Nano Formulation (Composition A) and Marketed Formulation (Non-Nano) Cream

In in-vitro skin retention studies, the drug is equilibrated on skin surface for about 1 hr followed by extraction by about 5 ml CH3CN/Buffer (8:2) using homogenization for about 5 min followed by sonication for about 10 min. Finally the sample is centrifuged; supernatant is filtered and subjected to ZOI (zone of inhibition) assay.

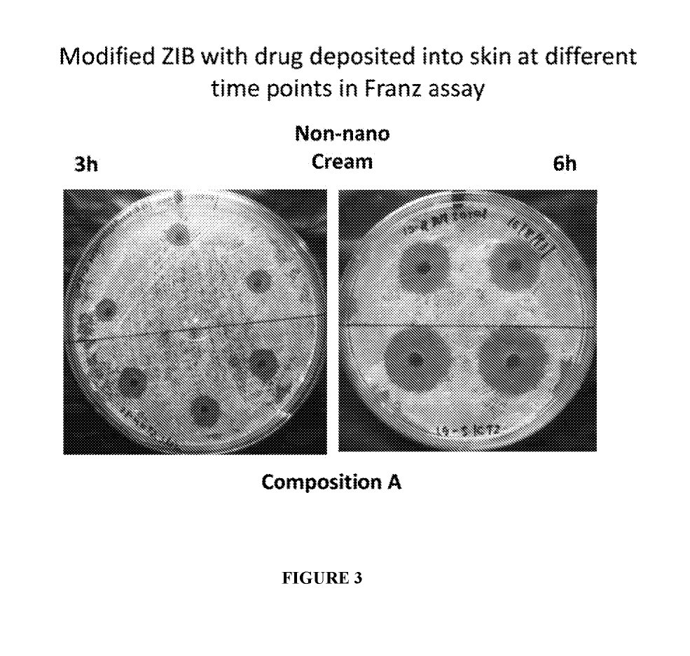

In this assay, about 100 μl of each sample is loaded into about 6 mm diameter wells in a SDA (Sabouraud dextrose agar) plate which is streaked with M. furfur strain and the results are depicted in FIG. 3.

FIG. 3 clearly shows higher ZOI of deposited drug present in Composition A formulation in comparison to the marketed cream. This further proves higher bio-activity of nano formulation as compared to non-nano formulation thus leading to enhanced fungal killing by ketoconazole emulsion gel (Composition A) of the present disclosure.

Further, in combination with FIG. 3, Table 4 depicts average ZOI values of deposited drug in skin after 3 h and 6 h contact time of drug into skin obtained from Franz assay and respective fungal killing efficacy for both non-nano cream and ‘instant disclosure emugel’ formulation.

TABLE 4

| | | Amount of | Total | | | |
|---|---|---|---|---|---|---|
| | | drug | amt of | Concentration | ZOI | |
| | | (ketoconazole) | drug | of drug | (average | Fungal |
| | | applied on the | deposited | deposited into | of 6 | killing |
| Time | Name | skin | into skin | skin | readings) | efficacy |
| 3 h | Non-nano | 1.3 mg | 10 μg | 1 μg/ml | 1.03 cm | 100% |
| | cream | | | | | |
| | Instant | 1.3 mg | 40 μg | 4 μg/ml | 1.6 cm | 155% |
| | disclosure | | | | | |
| | (Composition | | | | | |
| | A) | | | | | |
| 6 h | Non- | 1.3 mg | 32 μg | 3.2 μg/ml | 1.4 cm | 100% |
| | nanocream | | | | | |
| | Instant | 1.3 mg | 75 μg | 7.5 μg/ml | 1.8 cm | 130% |
| | disclosure | | | | | |
| | Emugel | | | | | |
| | (Composition | | | | | |
| | A) | | | | | |

### Example 4

### Preparation of PiroctoneOlamine Hair Cream NanoFormulation (Composition B)

Composition for preparation of piroctone olamine hair cream formulation with globule size in nano-range is provided in Table 5 below:

TABLE 5

| | | Quantity for 100 gm formulation | | | |
|---|---|---|---|---|---|
| | | Compo- | | Compo- | |
| Phases | Ingredients | sition B1 | | sition B2 | |
| Phase A | Piroctone olamine | 0.1 | g | 0.1 | g |
| | Propylene glycol | 3 | g | 3 | g |
| | monocaprylate | | | | |
| | Light Liquid Paraffin | 18 | g | 22 | g |
| | Cetyl alcohol | 2 | g | 2 | g |
| | Stearyl alcohol | 3 | g | 2.5 | g |
| | Ceteryl alcohol and glycoside | 2.5 | g | — | |
| | Steareth-2 | — | | 1 | g |
| | Steareth-21 | — | | 1.5 | g |
| Phase B | Polyvinyl Pyrollidone K90 | 1 | g | 1 | g |
| | Poloxamer 407 | 5 | g | 5 | g |
| | Di Sod. EDTA | 0.05 | g | 0.05 | g |
| | Polyethylene glycol 600 | 7.5 | g | 7.5 | g |
| | Propylene glycol | 3 | g | 3 | g |
| | Water | q.s | | q.s | |
| Phase C | Preservative | q.s | | q.s | |
| | Antioxidant | q.s | | q.s | |
| | Fragrance | q.s | | q.s | |
| | Silicone fluid | 1.0 | g | 1.0 | g |
| Phase D | Carbopol | 0.15 | g | 0.15 | g |

| | | |
|---|---|---|
| Triethanolamine | q.s | q.s |

Procedure for Preparation of Piroctone Olamine Hair Cream Formulation with Globule Size in Nano-Range:
(1) All the ingredients of phase A are added and heated to melt at temperature ranging from about 70° C. to about 75° C. Piroctone olamine is added and dissolved in oily phase.
(2) All the ingredients of phase B are mixed and stirred until poloxamer 407 gets dissolved and then Phase B is also heated at temperature ranging from about 70° C. to about 75° C. with continuous stirring (300-350 RPM).
(3) Phase A is added to Phase B with continuous stirring (550 RPM) at temperature of about 70° C.
(4) Ingredients of Phase C are added to pre-formed emulsion at room temperature with continuous stirring (700-800 RPM).
(5) Finally, Carbopol (phase D) is added and neutralized by triethanolamine and stirring is continued (850 RPM) until formulation becomes uniform and homogenous.

### Determination of Cream Droplet Size

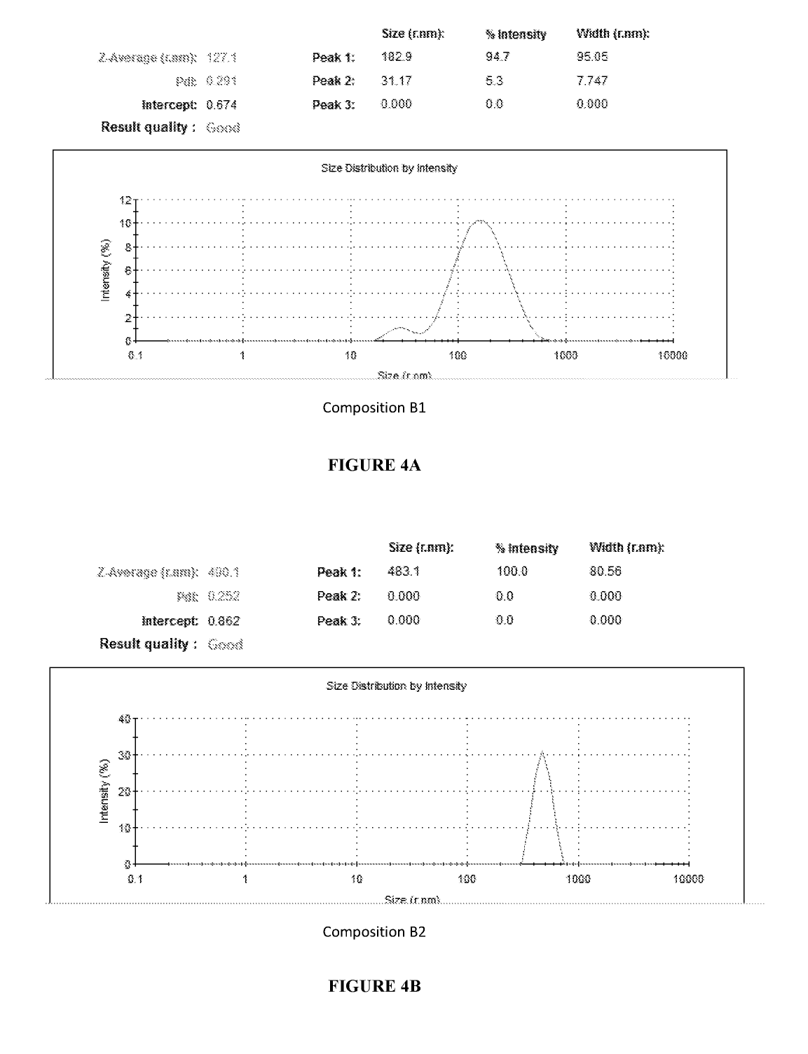

The mean droplet size of the emulsion is determined by dynamic light scattering (DLS) (Zetasizer, model ZS90, Malvern Instruments, UK). Globule size of Composition B1 is observed to be about 127 nm while Composition B2 shows globule size of approximately 490 nm as shown in FIG. 4A and FIG. 4B, respectively.

### Example 5

### Preparation of Piroctoneolamine Hair Gel NanoFormulation (Composition C)

Composition for preparation of piroctone olamine hair gel formulation with globule size in nano-range is provided as in Table 6 below:

TABLE 6

| | | Quantity (%) | |
|---|---|---|---|
| | | Compo- | Compo- |
| Phases | Ingredients | sition C1 | sition C2 |
| Phase A | Piroctoneolamine | 0.2 | 0.2 |
| | Polyoxyl 20 cetosteryl ether | 5 | 5 |
| | Propylene glycol monocaprylate | 2 | 2 |
| | PEG-8 Capric/Caprylate glyceride | 3 | 3 |
| | Water | 70.5 | 70.7 |
| Phase B | Polyethylene glycol 600 | 10 | 10 |
| | Ethanol | 5 | 5 |
| | Di Sod EDTA | 0.05 | 0.05 |
| | D-Panthenol | 0.25 | 0.25 |
| | Hydroxypropyl methyl cellulose | 0.2 | NA |
| | Preservative | q.s. | q.s. |
| | Fragrance | q.s. | q.s. |
| Phase C | Carbopol | 0.42 | 0.42 |
| | Triethanolamine | q.s | q.s |
| Phase D | Polyvinyl Pyrrolidone K90 | 3 | 3 |

Procedure for Preparation of Piroctone Olamine Hair Gel Formulation with Globule Size in Nano-Range
(1) All the ingredients of phase A are added and mixed at high stirring rate of about 600-700 RPM till the piroctone olamine is dissolved in Phase A (Surfactant Phase).
(2) All the ingredient of phase B are mixed and added to Phase A and stirred at RPM of about 200-300 until homogeneous phase is obtained.
(3) Phase C is added to above homogeneous mixture with continuous stirring and neutralized with triethanolamine until pH reaches to about 5.5 to about 6.0.
(4) Finally, Polyvinyl Pyrrolidone K90 is added and mixed at slow RPM till it gets mixed homogeneously.

### Determination of Gel Droplet Size

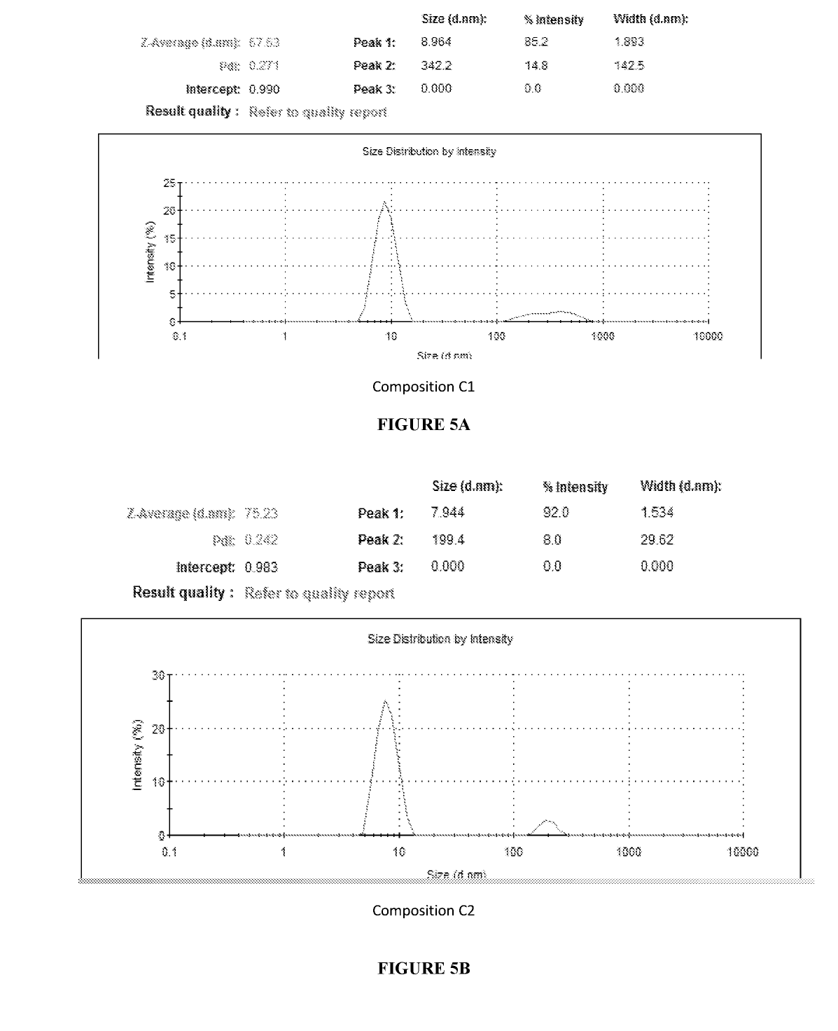

The mean droplet size of the gel is determined by dynamic light scattering (DLS) (Zetasizer, model ZS90, Malvern Instruments, UK). Globule size of Composition C1 is observed to be about 67.63 nm while Composition C2 has shown globule size of approximately 75.23 nm as shown in FIG. 5A and FIG. 5B, respectively.

### Example 6

### Preparation of Zinc Pyrithione Nanoparticles (Dispersions 1 & 2)

### Particle Nanotization

A required quantity of zinc pyrithione (ZPT; average particle size about 5000 nm) powder is added in portions to 1% aqueous solution of sodium docusate under stirring. The resulting suspension is passed through high pressure homogenizer at pressure of about 1200 bar to about 1500 bar. The output dispersion is collected in a beaker kept in ice bath and recycled about 6-10 times to yield a dispersion of appropriately sized particles (300 nm to 700 nm).

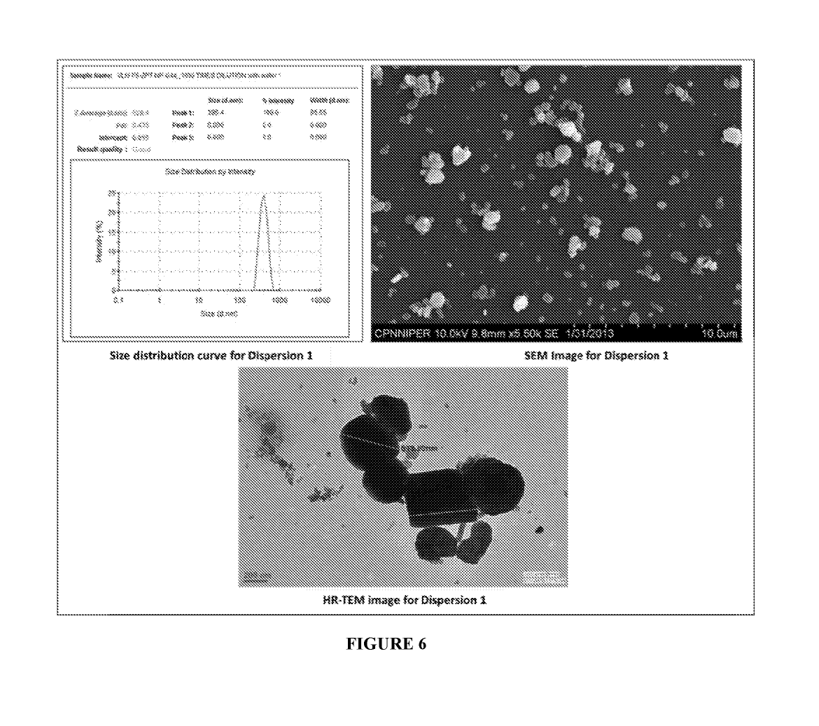

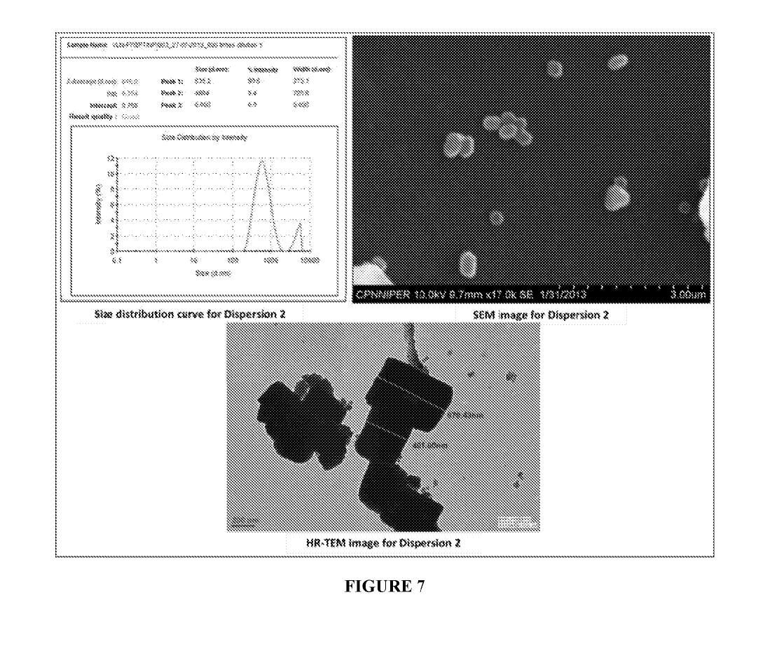

The size distribution and particle morphology are determined by ZetaSizer (ZS-90 from Malvern Instruments), Scanning Electron Microscope (SEM, Hitachi, S-3400 N, Japan), High-Resolution Transmission Electron Microscopy (HR-TEM, Tecnai G² F20 microscope; FEI, Eindhoven, The Netherlands) as shown in FIGS. 6 and 7.

### Dispersion Stabilization

The resulting dispersion is stabilized by addition of carbopol followed by neutralization with sodium hydroxide to a pH ranging from about 6.5 to about 7.0.

### Example 7

### Preparation of Zinc Pyrithione Hair Conditioner NanoFormulation (Composition D)

Conditioners with one or more nano-API(s) (zinc pyrithione of Example 6) and an oil component (fatty acid ester) consisting of carbon chain length less than 11 are designed and formulated as per the compositions shown in Table 7.

TABLE 7

| | | Composition D(%) | | |
|---|---|---|---|---|
| | | Compo- | Compo- | Compo- |
| | Strength | sition | sition | sition |
| Ingredient | (%) | D1 | D2 | D3 |
| Phase A | | | | |
| Water | NA | qs. | qs. | qs. |
| Carbopol | 30 | 3 | 3 | 2 |
| Sodium lauryl ether | 30 | 5 | 5 | 5 |
| sulphate | | | | |
| Sodium hydroxide | 18 | qs. | qs. | qs. |
| Phase B | | | | |
| Steareth-2 | NA | 3 | 3 | 3 |
| Steareth-21 | NA | 2 | 2 | 2 |
| Propylene glycol | NA | 9 | 9 | 3 |
| monocaprylate | | | | |
| Macrogolcetostearyl ether 20 | NA | 6 | 6 | 6 |
| Coco monoethanolamide | NA | 1.2 | 1.2 | 1.2 |
| Cetyl Alcohol | NA | 5 | 5 | 5 |
| Stearamidopropyl- | NA | 2 | 2 | 1 |
| dimethylamine | | | | |
| Lactic acid | 50 | qs. | qs. | qs. |
| Phase C | | | | |
| Cocamidopropylbetain | 30 | 10 | 10 | 10 |
| Cetrimoniumchloride | 30 | 2 | 2 | 2 |
| Polyquaternium-22 | NA | 0.5 | 0.5 | 0.5 |
| Amodimethicone emulsion | NA | 0.5 | 0.5 | 0.5 |
| Cassia hydroxypropyl- | 1 | 5 | 5 | 5 |
| trimoniumchloride | | | | |
| Propylene Glycol | NA | 2 | 2 | 2 |
| Glycerine | NA | 5 | 5 | 5 |
| Zinc pyrithione nano | 50*/10 | 1* | 1* | 10 |
| dispersion | | | | |
| Zinc Carbonate | NA | 1 | 1 | 1 |
| Titanium Dioxide | NA | 0.5 | 0.5 | 0.5 |
| Linalool | NA | 1 | 1 | 1 |
| Fragrance | NA | qs. | qs. | qs. |
| Chloromethyl/ | 1.51 | 0.05 | 0.05 | 0.05 |
| Methylisothiazolinone | | | | |

### Method of Preparation:

(1) Phase A: A required amount of water is added to a mixing vessel and stirred slowly (50-55 rpm) using an overhead stirrer. Carbopol is added to water followed by the slow addition of about 30% aqueous solution of sodium lauryl ether sulfate (SLES). Then mixture is neutralized by adding sodium hydroxide solution.
(2) Phase B: Components of Phase B are mixed and heated to melt. Lactic acid is added to the resulting melted mixture to neutralize. The Phase B is added to Phase A while stirring at about 60° C. After uniform mixing, the mixture is allowed to cool to 35° C. to 40° C.
(3) Phase C: To the above stirring mixture, cocamidopropylbetaine, cetrimonium chloride, polyquaternium-22, amodimethicone emulsion, Cassia hydroxypropyltrimonium chloride, propylene glycol and glycerin are added slowly in the same order as mentioned in Tableland stirred (50-100 rpm) till uniform mixing. Zinc pyrithione fine particle suspension (ZPT FPS) or ZPT nanoparticles (ZPT NPs) dispersion is added to the stirring mixture followed by addition of zinc carbonate and titanium dioxide. The mixture is then allowed to cool to room temperature. Finally, linalool, fragrance and preservatives are added, and the mixture is allowed to stir in order to yield a smooth uniform conditioner cream (about 150-160 rpm).

### Example 8

### Preparation of Nano Zincpyrithione Based Shampoo Formulations (Composition E)

Shampoo formulations with one or more nano-API(s) (zinc pyrithione of Example 6) and an oil component (fatty acid ester) consisting of carbon chain length less than 11 were designed and formulated as per the compositions shown in Table 8.

TABLE 8

| | | Composition in % | | |
|---|---|---|---|---|
| | Strength | Exam- | Exam- | Exam- |
| Ingredient | in % | ple-4 | ple-5 | ple-6 |
| Phase A | | | | |
| Water | NA | qs. | qs. | qs. |
| Carbopol | NA | 2.5 | 2.5 | 2.5 |
| Ammonium lauryl sulfate | 30 | 10 | 10 | 10 |
| Sodium lauryl ether sulfate | 30 | 30 | 30 | 30 |
| Sodium hydroxide | 18 | qs. | qs. | qs. |
| Phase B | | | | |
| Coco monoethanolamide | NA | 1 | 1 | 1 |
| Cetyl alcohol | NA | 0 | 4 | 0 |
| Proylene glycol monocaprylate | NA | 4 | 0.1 | 4 |
| Menthol | NA | 0.2 | 0.1 | 0.1 |
| Phase C | | | | |
| Zinc pyrithione nano | 10/NA* | 10 | 10 | 1* |
| dispersion/powder* | | | | |
| Magnessium sulfate | NA | 0.5 | 0.5 | 0.5 |
| Amodimethicone | NA | 1 | 1 | 1 |
| Cetrimoniumchloride | NA | 1 | 0 | 0 |
| Propylene glycol | NA | 1 | 1 | 1 |
| Zinc carbonate | NA | 1 | 1 | 1 |
| Cocamidopropylbetaine | 30 | 10 | 10 | 10 |
| Cassia hydroxypropyl- | 1 | 10 | 10 | 10 |
| trimoniumchloride | | | | |
| Chloromethyl/ | 1.51 | 0.05 | 0.05 | 0.05 |
| Methylisothiazolinone | | | | |
| Fragrance | NA | qs. | qs. | qs. |
| Citric acid | 50 | qs. | qs. | qs. |
| Sodium chloride | 30 | qs. | qs. | qs. |

### Method of Preparation:

(1) Phase A: A required amount of water is added to a mixing vessel and stirred slowly (50-55 rpm) using an overhead stirrer. Carbopol is added to water followed by the slow addition of a premix of about 30% aqueous solutions of ammonium lauryl sulfate (ALS) and sodium lauryl ether sulfate (SLES). The mixture was neutralized by sodium hydroxide solution.
(2) Phase B: A mixture of CMEA (cocamidemonoethanolamide), menthol and propylene glycol monocaprylate is heated to melt. The resulting melt is immediately poured to Phase A while stirring at about 60° C. After stirring for about 5 min at the same temperature, it is allowed to cool to about 35° C. to about 40° C.
(3) Phase C: ZPT NPs (or ZPT powder as control) dispersion is added to the above stirring mixture. Then, magnesium sulfate is added while stirring followed by additions of amodimethicone emulsion and propylene glycol, followed by the addition of zinc carbonate, cocamidopropylbetaine (30% aq.), cassia hydroxypropyltrimonium chloride and preservatives in the same order as mentioned in Table 8. The continuously stirring mixture (150-160 rpm) is then allowed to cool to room temperature followed by addition of fragrance. Finally, pH is adjusted with citric acid and viscosity by sodium chloride, and mixture is continued to be stirred to yield a smooth and shiny shampoo (maximum speed of about 150-160 rpm).

### Dose Response Curves (Using Zones of Inhibition) of Nano ZPT Dispersion Versus Commercial ZPT Powder

Agar well-diffusion method was employed to run Zone of Inhibition (ZOI) assays. ZOI values may vary for compounds having different diffusion coefficients. ZOI was employed to assess the potency of API and/or formulation to inhibit the growth of microorganisms under study. ZOI values, determined at different API concentrations, can be used to derive dose-response-curves (DRCs) for efficacy comparison of different APIs/formulations.

### Method:

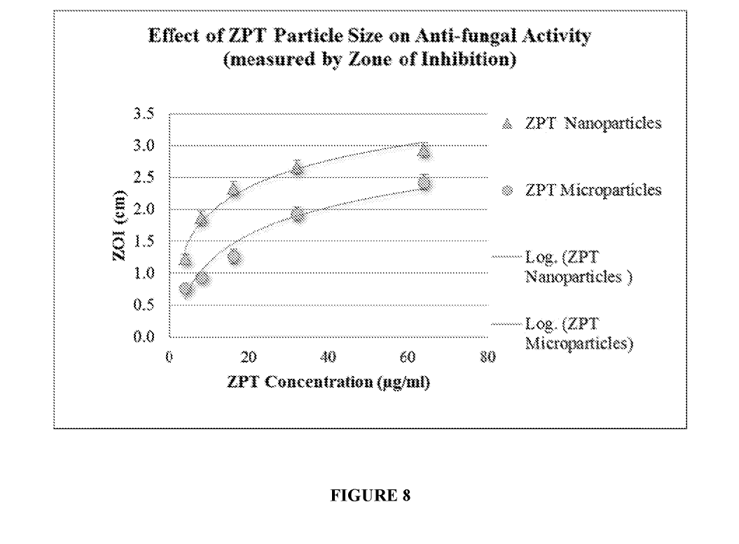

Malassezia furfur culture of 1-7×10⁶ cfu/ml was used to inoculate Sabaroud's Dextrose agar (SDA) plates [supplemented with chloramphenicol (0.25 mg/ml), cycloheximide (0.04 mg/ml) and olive oil (2%)]. Approximately, 6 mm wells were created in the agar plate using sterile straws. The wells were supplemented with different concentrations (4-64 μg/ml) of ZPT nanoparticles and micro particles (commercially purchased) and/or controls (100 μl each). The plates then were incubated at 30±2° C. under CO₂ (5%) atmosphere. Readouts were taken after 42 hrs or 72 hrs. An example of effect of particle size (ZPT particles) on anti-fungal activity (measured using Zone of Inhibition studies) was demonstrated in FIG. 8.

It was evident from the graph depicted in the FIG. 8, that the ZPT nanoparticles showed more growth inhibition when compared to the commercially procured ZPT powder which is microparticular in nature, thereby showcasing the enhanced efficacy of nano-ZPT particles of the present disclosure.

### Example 9

### Preparation of Antiviral Acyclovir Cream NanoFormulation (Composition F)

The antiviral cream with globular size in nano-range is prepared in similar way as described in Example 4. Composition for preparation acyclovir cream formulation with globule size in nano-range is provided in Table 9 below:

TABLE 9

| | | Quantity for 100 gm formulation | |
|---|---|---|---|
| Phases | Ingredients | Composition B1 | |
| Phase A | Acyclovir | 5.0 | g |
| | Propylene glycol | 3 | g |
| | monocaprylate | | |
| | Light Liquid Paraffin | 23 | g |
| | Cetyl alcohol | 2 | g |
| | Stearyl alcohol | 3 | g |
| | Ceteryl alcohol and glycoside | 2.5 | g |
| | Steareth-2 | — | |
| | Steareth-21 | — | |
| Phase B | Polyvinyl Pyrollidone K90 | 1 | g |
| | Poloxamer 407 | 5 | g |
| | Di Sod. EDTA | 0.05 | g |
| | Polyethylene glycol 600 | 7.5 | g |
| | Propylene glycol | 3 | g |
| | Water | q.s | |
| Phase C | Preservative | q.s | |
| | Antioxidant | q.s | |
| | Fragrance | q.s | |
| | Silicone fluid | 1.0 | g |
| Phase D | Carbopol | 0.15 | g |

| | |
|---|---|
| Triethanolamine | q.s |

Procedure for Preparation of Antiviral Acyclovir Cream Formulation with Globule Size in Nano-Range
(1) All the ingredients of phase A are added and heated to melt at temperature ranging from about 70° C. to about 75° C. Acyclovir is added and dissolved in oily phase.
(2) All the ingredient of phase B are mixed and stirred until poloxamer 407 gets dissolved and then Phase B is also heated at temperature ranging from about 70° C. to about 75° C. with continuous stirring (300-350 RPM).
(3) Phase A is added to Phase B with continuous stirring (550 RPM) at temperature of about 70° C.
(4) Ingredients of Phase C are added to pre-formed emulsion at room temperature with continuous stirring (700-800 RPM).
(5) Finally, Carbopol (phase D) is added and neutralized by triethanolamine and stirring is continued (850 RPM) until formulation becomes uniform and homogenous.

### Determination of Cream Droplet Size

The mean droplet size of the emulsion is determined by dynamic light scattering (DLS) (Zetasizer, model ZS90, Malvern Instruments, UK).

### Example 10

### Preparation of Antiviral Penciclovir Emulsion Gel Nano Formulation (Composition G)

The antiviral Penciclovir emulsion gel is prepared as described in Example 1 Composition for preparation of Penciclovir emulsion gel is given as in Table 10 below:

TABLE 10

| | |
|---|---|
| Ingredient | Quantity (%) |

Phase A

| | |
|---|---|
| Penciclovir | 1.0 |
| Lauryl alcohol | 4.3 |
| Propylene glycol monocaprylate | 3.3 |
| Steareth-2 | 3.3 |
| Steareth-21 | 3.3 |

Phase B

| | |
|---|---|
| Poloxamer | 6.5 |
| Glycerine | 3.0 |
| Water | qs. |

Phase C

| | |
|---|---|
| Preservative | qs. |
| Fragrance | qs. |

Phase D

| | |
|---|---|
| pH Modifier | qs. |

### Procedure for Preparation of Penciclovir Emulsion Gel Nano-Formulation

(1) Phase A: 10 mg Penciclovir is solubilized in a mixture containing 43 mg Lauryl alcohol, 33 mg Sefsol 218, 33 mg Steareth 2 and 33 mg Steareth 21. Poloxamer (65 mg) is added into the mixture and the temperature is maintained at about 70-80° C.
(2) Phase B: Water phase contains 30 mg Glycerine and temperature is maintained at about 70° C. to about 80° C.
(3) Phase (A) is homogenized with phase (B) upon stirring (at about 700 rpm) and cooled to a temperature of about 35° C. to about 40° C. to obtain Phase C.
(4) The fragrance and preservative is added into Phase C while stirring at about 500 rpm, to obtain Phase D.
(5) 18% sodium hydroxide is added into Phase D to maintain pH ranging from about 5.0 to about 6.0.
(6) The pH, viscosity and globule size at different water dilution is measured.

### Measurement of the Globule Size

The globule size is measured by Malvern Zetasizer using dynamic light scattering (DLS) technique. It is further confirmed by Transmission electron microscope (TEM) and Scanning Electron Microscope (SEM) studies.

### Example 11

### Preparation of Triclosan Nanoparticles (Dispersions X1 & X2)

### Particle Nanotization:

A required quantity of Triclosan (TCN; average particle size about 6000 nm) powder is added in portions to 1% aqueous solution of sodium docusate under stirring. The resulting suspension is passed through high pressure homogenizer at pressure of about 1300 bar to about 1600 bar. The output dispersion is collected in a beaker kept in ice bath and recycled about 6-10 times to yield a dispersion of appropriately sized particles (200 nm to 700 nm). The size distribution is determined by ZetaSizer (ZS-90 from Malvern Instruments) and Scanning Electron Microscope (SEM, Hitachi, S-3400 N, Japan).

### Dispersion Stabilization

The resulting dispersion is stabilized by addition of carbopol followed by neutralization with sodium hydroxide to a pH ranging from about 6.0 to about 6.5.

### Example 12

### Preparation of Triclosan General Purpose Antimicrobial (Antibacterial+Antifungal) Cream NanoFormulation (Composition G)

Creams with one or more nano-API(s) of triclosan (Example X) and an oil component (are designed and formulated as per the compositions shown in Table 11.

TABLE 11

| | Strength | Composition in % | |
|---|---|---|---|
| Ingredient | (%) | Composition G1 | Composition G2 |
| Phase A | | | |
| Water | NA | qs. | qs. |
| Carbopol Ultrez 20 | NA | 1 | 1 |
| NaOH | 30 | 0.2 | 0.2 |

Phase B

| | | | |
|---|---|---|---|
| Cylcopentyl Siloxane | 96 | 5 | 4 |
| Laureth-4 | NA | 0.45 | 0.45 |
| Laureth-23 | NA | 0.09 | 0.09 |
| Steareth-2 | NA | 2.46 | 2.46 |

Phase C

| | | | |
|---|---|---|---|
| Glycerol | 30 | 5 | 6 |
| Dispersion X1/X2 | 25 | 1 (X1) | 1 (X2) |
| Vit. E | NA | 0.5 | 0.5 |
| Vit. B5 | NA | 0.5 | 0.5 |
| EDTA | NA | 0.05 | 0.05 |
| Propyl Paraben | NA | 0.3 | 0.3 |
| Methyl Paraben | NA | 0.03 | 0.03 |
| Color | NA | qs | qs |
| Fragrance | NA | qs | qs |

### Method of Preparation:

(1) Phase A: A required amount of water is added to a mixing vessel and stirred slowly (50-55 rpm) using an overhead stirrer. Carbopol is added to water and stirred for about 20-25 min so as to allow carbopol to swell. This is followed by neutralization using sodium hydroxide solution. The mixture is slowly heated under stirring condition to reach the temperature of about 65° C. to 70° C.
(2) Phase B: Components of Phase B are mixed and heated to melt. The Phase B is added to Phase A while stirring at about 65° C. to 70° C. After uniform mixing, the mixture is allowed to cool to 35° C. to 40° C. with continuous stirring at about 200 rpm.
(3) Phase C: To the above stirring mixture, contents of Phase C, except fragrance, are added one-by-one serially, and the resulting mixture is stirred at about 300-400 rpm to ensure uniform mixing. The mixture is then allowed to cool to room temperature. Finally, fragrance is added, and the mixture is allowed to stir in order to yield a smooth uniform cream formulation (about 300-400 rpm).

### Example 13

### Effect of Propylene Glycol Monocaprylate Addition (Below C11 Fatty Acid Ester) on Zinc Pyrithione Activity Towards Malassezia furfur

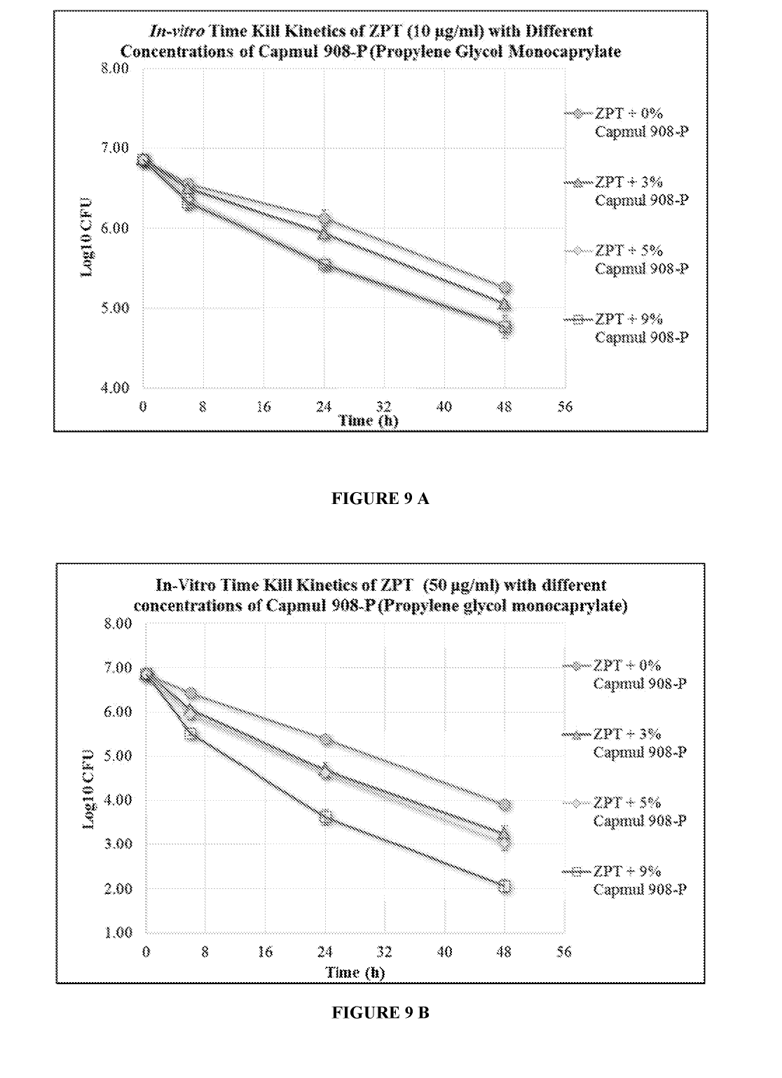

An activity comparison, by in vitro time-kill kinetics of zinc pyrithione using plain zinc pyrithione (Kopithione, Kumar Organic Products Ltd) versus its combination with different concentrations of propylene glycol monocaprylate (Capmul 908-P, Croda), was demonstrated. (FIGS. 9A and (9B)

The time-kill assays were used to evaluate efficacy of antimicrobial agents, either singly or in combination, and the results can help in establishing the dose and/or time of application of the active. The time-kill assays were used to study both concentration-dependent and time-dependent antimicrobial action.

### Method:

M. furfur cells were suspended in Sabouraud Dextrose Broth (SDB) at inoculum concentration of 7×10⁷ cells/ml. Cells were taken from a freshly growing (3-7 days old) plate and cell suspension was vortexed to remove the cell clumps as much as possible. Sterile media were supplemented with chloramphenicol (0.25 mg/ml), cycloheximide (0.04 mg/ml) and olive oil (2%). The media were then supplemented with appropriate concentrations (two-fold serial dilutions using SDB) of unmodified zinc pyrithione API (10 μg/ml and 50 μg/ml) with different concentrations of Capmul 908-P (0%, 1%, 3%, 5% & 9%). The cultures were incubated on a tube rotator at 34° C. in CO₂ incubator.

To measure the colony forming units (CFU), at different time points, aliquots (50 μl) of Malassezia cultures were serially diluted with SDBT medium (SDB containing 0.1% Triton X-100) and plated on SDA plates. The plates are incubated at 34° C. in CO₂ incubator for 3 days. The viable colonies were counted and converted to CFU/ml. The results of time kill study using zinc pyrithione powder with different concentration of Capmul 908-P are shown in Tables 12 and 13 and are plotted in FIGS. 9A and 9B.

The results of the assays suggested that the anti-microbial activity of zinc pyrithione towards M. furfur, gets potentiated with increasing concentration of propylene glycol monocaprylate (Capmul 908-P).

TABLE 12

| | Log CFU Count of M. furfur at ZPT concentration | | | |
|---|---|---|---|---|
| | of 10 μg/ml with different Capmul 908-P concentration | | | |
| Time | Capmul 908-P | Capmul 908-P | Capmul 908-P | Capmul 908-P |
| (hrs) | (nil) | (30 μg/ml) | (50 μg/ml) | (90 μg/ml) |
| 6 | 6.56 | 6.50 | 6.37 | 6.33 |
| 24 | 6.13 | 5.94 | 5.57 | 5.55 |
| 48 | 5.27 | 5.06 | 4.80 | 4.77 |

TABLE 13

| | Log CFU Count of M. furfur at ZPT concentration | | | |
|---|---|---|---|---|
| | of 50 μg/ml with different Capmul 908-P concentration | | | |
| Time | Capmul 908-P | Capmul 908-P | Capmul 908-P | Capmul 908-P |
| (h) | (nil) | (30 μg/ml) | (50 μg/ml) | (90 μg/ml) |
| 6 | 6.43 | 6.06 | 5.94 | 5.52 |
| 24 | 5.39 | 4.69 | 4.62 | 3.63 |
| 48 | 3.92 | 3.25 | 3.04 | 2.08 |

### Example 14

### Comparative Efficacy of Nano Composition Having C<11 and Non-Nano Composition Having C>10

To substantiate the efficacy of the instant disclosure, a comparative study is conducted wherein an ex-vivo pig skin model is used for showcasing fungal inhibition. The freshly excised pig ear skin is taken and 10 mg of each formulation are applied in an area of 4 cm over a period of time. The various formulations applied are as follows:
a) Non-Nano Formulation with C>10 fatty acid/ester.
b) Nano-Formulation or Instant emugel Formulation with C<11 fatty acid/ester.
c) Nano-Formulation without C<11 fatty acid or ester.

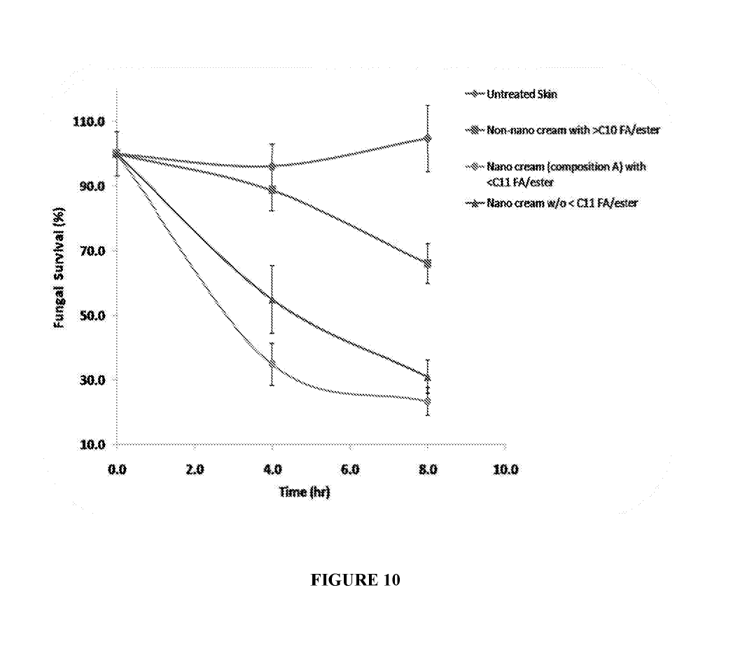

As shown graphically in FIG. 10, the results indicate high percentage of fungal inhibition by ‘Instant emugel Formulation’ which contains a <C11 agent (Sefsol) and is devoid of C>10FAs/esters in comparison to non-nano formulation with C>10FAs/esters. This ex-vivo fungal assay further proves the importance of addition of a C<11 agent in the nano-formulation devoid of components with >C10 FAs/esters.

Both of these features, i.e. the presence of C11 agent and absence of components devoid of C>10 FAs/esters play a highly significant role in formulating an effective topical antifungal formulation with 2% ketoconazole.

### Example 15

### Leave-on Emugel Nano Composition with Besifloxacin for Acne Treatment

Fluoroquinolones are broad-spectrum antibiotics (effective for both gram-negative and gram-positive bacteria) that play an important role in treatment of serious bacterial infections. The composition of anti-acne for face or body as leave-on emugel system is described below:

TABLE 14

| | |
|---|---|
| Ingredients | % Use |

Phase A

| | |
|---|---|
| Besifloxacin•HCl | 1.0 |
| Propylene glycol monocaprylate | 5.0 |
| Cetyl alcohol | 1.0 |
| PEG 2 stearyl ether | 2.0 |
| PEG 21 stearyl ether | 2.0 |
| Poloxamer 407 | 7.0 |

Phase B

| | |
|---|---|
| Propylene glycol | 3.0 |
| Water | q.s. to 100.0 |

Phase C

| | |
|---|---|
| Acrylates C-10-30 alkyl acrylate crosspolymer | 0.1 |

Phase D

| | |
|---|---|
| Antioxidant | 0.1 |
| Preservative | 0.5 |

Phase E

| | |
|---|---|
| pH modifier | q.s. to pH 5-6 |

### Method of Preparation:

1) Phase A ingredients are mixed together in a glass beaker and heated upto 60-70° C.
2) Phase B ingredients are mixed together in a glass beaker and heated upto 60-70° C.
3) Phase A is added into phase B slowly, with continuous stirring at 500 rpm.
4) Resulting emulsion was cooled upto 40° C. with continuous stirring at 500 rpm.
5) Phase C is added to the emulsion with continuous stirring at 700 rpm.
6) Phase D is mixed together and added to the emulsion with continuous stirring at 700 rpm.
7) Finally pH of the formulation is adjusted with phase E.

### Example 16

### Leave-on Emugel Composition with Besifloxacin-Adapalene Combination for Acne Treatment

The composition of anti-acne for face or body as leave-on emugel system comprising besifloxacin and adapalene is described below:

TABLE 15

| | |
|---|---|
| Ingredients | % Use |

Phase A

| | |
|---|---|
| Besifloxacin HCl | 1.0 |
| Adapalene | 0.1 |
| Propylene glycol monocaprylate | 5.0 |
| Cetyl alcohol | 1.0 |
| PEG 2 stearyl ether | 2.0 |
| PEG 21 stearyl ether | 2.0 |
| Vitamin E polyethylene glycol succinate | 2.0 |
| Poloxamer 407 | 7.0 |

Phase B

| | |
|---|---|
| Propylene glycol | 3.0 |
| Water | q.s. to 100.0 |

Phase C

| | |
|---|---|
| Antioxidant | 0.1 |
| Preservative | 0.5 |

Phase D

| | |
|---|---|
| pH modifier | q.s. to pH 5-6 |

### Method of Preparation:

1) Phase A ingredients are mixed together in a glass beaker and heated upto 60-70° C.
2) Phase B ingredients are mixed together in a glass beaker and heated upto 60-70° C.
3) Phase A is added into phase B with continuous stirring at 500 rpm.
4) Resulting emulsion is cooled upto 400 C, with continuous stirring at 500 rpm.
5) Phase C ingredients are mixed together and added to the emulsion with continuous stirring at 700 rpm.
6) Finally pH of the formulation is adjusted with phase D.

### Example 17

### Combination of Ketoconazole and Piroctone Olamine

The composition with the combination of ketoconazole and piroctone olamine is described in the below table:

TABLE 16

| | |
|---|---|
| Ingredients | % used |

Phase A

| | |
|---|---|
| KTZ | 2.0% |
| Piroctone Olamine | 0.1% |
| Lauryl alcohol | 3.3% |
| Caproyl 90 | 3.3% |
| Steareth 2 | 3.3% |
| Steareth 21 | 3.3% |

Phase B

| | |
|---|---|
| Poloxamer 407 | 7.0% |
| Propylene glycol | 3.0% |
| Water | 74.1% |

Phase C

| | |
|---|---|
| Antioxidant | 0.1% |
| Preservative | 0.5% |

Phase D

| | |
|---|---|
| pH modifier | q.s. |

### Method of Preparation:

1) Phase A: 20 mg Ketoconazole and 1.0 mg Piroctone Olamine is solubilized in a mixture containing 33 mg Lauryl alcohol, 33 mg Capryol 90, 33 mg Steareth 2 and 33 mg Steareth 21. Poloxamer (70 mg) is added into the mixture and the temperature is maintained at about 70-80° C.
2) Phase B: Water phase contains 30 mg Propylene glycol and temperature is maintained at about 70° C. to about 80° C.
3) Phase (A) is homogenized with phase (B) upon stirring (at about 700 rpm) and cooled to a temperature of about 35° C. to 40° C. to obtain Phase (C).
4) The antioxidant and preservative is added into Phase (C) while stirring the mixture at about 500 rpm, to obtain Phase (D).
5) Citric acid is added into Phase (D) to maintain pH ranging from about 5.0-6.0.

### Example 18

### Combination of Ketoconazole (KTZ) and Salicylic Acid

The composition with the combination of ketoconazole and salicylic acid is described in the below table:

TABLE 17

| | |
|---|---|
| Ingredients | % used |

Phase A

| | |
|---|---|
| KTZ | 2.0% |
| Salicylic acid | 0.5% |
| Lauryl alcohol | 3.3% |
| Caproyl 90 | 3.3% |
| Steareth 2 | 3.3% |
| Steareth 21 | 3.3% |

Phase B

| | |
|---|---|
| Poloxamer 407 | 7.0% |
| Propylene glycol | 3.0% |
| Water | 73.7% |

Phase C

| | |
|---|---|
| Antioxidant | 0.1% |
| Preservative | 0.5% |

Phase D

| | |
|---|---|
| pH modifier | q.s. |

### Method of Preparation:

1) Phase A: 20 mg Ketoconazole and 5.0 mg Salicylic acid is solubilized in a mixture containing 33 mg Lauryl alcohol, 33 mg Capryol 90, 33 mg Steareth 2 and 33 mg Steareth 21. Poloxamer (70 mg) is added into the mixture and the temperature is maintained at about 70-80° C.
2) Phase B: Water phase contains 30 mg Propylene glycol and temperature is maintained at about 70° C. to 80° C.
3) Phase (A) is homogenized with phase (B) upon stirring the mixture at about 700 rpm and cooled to a temperature of about 35° C.-40° C. to obtain Phase (C).
4) The antioxidant and preservative is added into Phase (3) while stirring at about 500 rpm, to obtain Phase (D).
5) Sodium hydroxide is added into Phase (D) to maintain pH ranging from about 5.0 to about 6.0.

We claim:

1. An anti-microbial composition comprising:
a) at least one anti-microbial agent;
b) optionally at least one oil, or a fatty acid or ester thereof, or both; and
c) at least one excipient;
wherein said fatty acid or ester thereof is having less than 11 carbon atoms; wherein the composition is devoid of fatty acids or esters having more than 10 carbon atoms; and
wherein particle size of at least one component is in nano-scale range.

2. The anti-microbial composition as claimed in claim 1, wherein the component having particle size in the nano-scale range is the anti-microbial agent.

3. The anti-microbial composition as claimed in claim 1, wherein the composition is formulated in a manner wherein, particle size or globule size of the formulation is in nanoscale range of about 1 nm to about 10,000 nm; preferably in the range of about 10 nm to about 1000 nm.

4. The anti-microbial composition as claimed in claim 3, wherein the formulation is a cream, oil, lotion, serum, gel, shampoo, nail varnish, ointment, foam, spray, conditioner, paste, mouthwash, sanitizer, solution, patch or aerosol.

5. The anti-microbial composition as claimed in claim 1, wherein the anti-microbial agent is at a concentration ranging from about 0.01% to about 50% by weight of the total composition; preferably at a concentration ranging from about 0.01% to about 10% by weight of the total composition; and more preferably at a concentration ranging from about 0.01% to about 5% by weight of the total composition.

6. The anti-microbial composition as claimed in claim 1, wherein the anti-microbial agent is selected from a group comprising anti-fungal agent, anti-bacterial agent and anti-viral agent or any combination thereof.

7. The anti-microbial composition as claimed in claim 6, wherein the anti-fungal agent is selected from a group comprising piroctoneolamine, ciclopiroxolamine, ketoconazole, climbazole, miconazole nitrate, itraconazole, fluconazole, econazole, terconazole, saperconazole, amorolfine, oxiconazole, clotrimazole, luliconazole, terbinafine, butenafine, naftifine, selenium disulfide, salicylic acid, sulfur, tar, undecanoic acid, zinc pyrithione, hinokitol, arnica extract, walnut shell extract, tea tree oil, rosemary oil and birch oil or any combination thereof.

8. The anti-microbial composition as claimed in claim 6, wherein the anti-bacterial agent is selected from a group comprising, macrolides, ketolides, beta lactams, monolactams, quinolones, sulfonamides, sulphathalidine, aminoglycosides, tetracyclines, rifamycins, glycopeptides, streptogramins, oxazolidinones, polymyxin, colistin, colymycin, trimethoprim, bacitracin, triclosan, besifloxacin, plurifloxacin, ornidazole, cephalothin, cefoxitin and phosphonomycin or any combination thereof.

9. The anti-microbial composition as claimed in claim 6, wherein the anti-viral agent is selected from a group comprising Acylovir, Imiquimod, Docosanol, Penciclovir, Podophyllin, Podofilox, Aciclovir, Adefovir, Amantadine, Amprenavir, Arbidol, Atazanavir, Balavir, Boceprevirertet, Cidofovir, Combivir, Darunavir, Delavirdine, Didanosine, Edoxudine, Efavirenz, Emtricitabine, Enfuvirtide, Entecavir, Famciclovir, Fomivirsen, Fosamprenavir, Foscarnet, Fosfonet, Ganciclovir, Ibacitabine, Imunovir, Idoxuridine, Indinavir, Inosine, Integrase inhibitor, Lamivudine, Lopinavir, Loviride, Maraviroc, Moroxydine, Methisazone, Nelfinavir, Nevirapine, Nexavir, Nucleoside analogues, Oseltamivir, Peginterferon alfa-2a, Penciclovir, Peramivir, Pleconaril, Podophyllotoxin, Protease inhibitor, Raltegravir, Reverse transcriptase inhibitor, Ribavirin, Rimantadine, Ritonavir, Pyramidine, Saquinavir, Stavudine, Telaprevir, Tenofovir, Tenofovirdisoproxil, Tipranavir, Trifluridine, Trizivir, Tromantadine, Truvada, Valaciclovir, Valganciclovir, Vicriviroc, Vidarabine, Viramidine, Zalcitabine, Zanamivir, and Zidovudine or any combination thereof.

10. The anti-microbial composition as claimed in claim 1, wherein the oil is either devoid of fatty acid or ester thereof or the oil comprises fatty acid or ester having less than 11 carbon atoms.

11. The anti-microbial composition as claimed in claim 1, wherein the oil is selected from a group comprising paraffin oil, silicone oil, terpene, fatty alcohol, dibutyladipate, dioctyladipate, cetyl alcohol, stearyl alcohol and ceteryl alcohol or any combination thereof.

12. The anti-microbial composition as claimed in claim 1, wherein the fatty acid or the ester thereof having less than 11 carbon atoms is selected from a group comprising propionic acid, butyric acid, pentanoic acid, hexanoic acid, heptanoic acid, caprylic acid, nonanoic acid, capric acid, mono or di ester of said acid with propylene glycol and mono or di or tri esters of said acid with glycerol, or any combination thereof; and wherein the fatty acid or the ester thereof is a part of the oil or an independent fatty acid or ester thereof.

13. The anti-microbial composition as claimed in claim 1, wherein the oil or the fatty acid or the ester thereof is at a concentration ranging from about 0.5% to about 99% by weight of the total composition; preferably at a concentration ranging from about 50% to about 99% by weight of the total composition; more preferably at a concentration ranging from about 0.5% to about 20% by weight of the total composition.

14. The anti-microbial composition as claimed in claim 1, wherein the excipient is selected from a group comprising active agent, solvent, emulsifier, surfactant, polymer, stabilizer, oil and additive or any combination thereof.

15. The anti-microbial composition as claimed in claim 14, wherein the active agent is selected from a group comprising pharmaceutical active, OTC active, anti-inflammatory agent and skin penetration enhancer or any combination thereof; solvent is selected from a group comprising C-1 to C-6 lower aliphatic alcohols, lower alkyl acetate, ethers, carboxylic acid, derivatives containing carbon chain length less than C11 and fatty alcohols or any combination thereof; wherein the emulsifier is selected from a group comprising steareth-2, Steareth-21, Poloxamer, Macrogolcetostearyl ether 20, cetyl alcohol cetearths, ceteth, isoceteths, laureths, oleths, steareths, lauramide DEA, and linoleamide DEA or any combination thereof; wherein the surfactant is selected from a group comprising Poloxamer, PEG-2 stearyl ether, PEG-21 stearyl ether, Pluoronic F127 (poloxamer), Polyoxyl 20 cetosteryl ether, Sodium laryl ether sulphate, Coco monoethanolamide, Cocamidopropylbetain, Sodium docusate and Ammonium lauryl sulphate or any combination thereof; and wherein the additive is selected from a group comprising, thickeners, antioxidants, perfumes or fragrances, essential oils, pH adjusters, herbal extracts, preserving agents, hair conditioning substances, hair care adjuncts, skin care adjuncts, emollient, dyestuffs, moisturizers, vitamins, sphingoceryls, sunscreens, co-surfactants, foaming agents, co-emulsifiers, viscosity modifiers, suspending agents, potentiating agents, pearlizing agents, cooling agents, ionic strength modifiers and oil-soluble polymers which are compatible with the base oil or skin care agents or both including skin-nutrient agents, anti-wrinkle agents, light and dust protectors or any combination thereof.

16. The anti-microbial composition as claimed in claim 1, wherein the excipient is at a concentration ranging from about 0.5% to about 99.90% by weight of the total composition.

17. A process for obtaining an anti-microbial composition as claimed in claim 1, said process comprising act of: combining at least one anti-microbial agent with at least one excipient, optionally along with at least one oil, or a fatty acid or ester thereof, or both, in a manner such that at least one component has a particle size in nano-scale range; and wherein the composition is devoid of fatty acids or esters having more than 10 carbon atoms.

18. The process as claimed in claim 17, wherein the component is subjected to nanotization prior to the combining, or wherein the combination is subjected to homogenization to obtain the composition having the at least one component having a particle size in nano-scale range.

19. The process as claimed in claim 18, wherein the homogenization of the combination results in in-situ generation of the nano-scale particles during the process for obtaining the composition.

20. The process as claimed in claim 18, wherein the nanotization is carried out by a process comprising acts of:
a) combining the at least one component with a surfactant under stirring to obtain a suspension;
b) passing the resultant suspension through a homogenizer at high pressure and collecting the output dispersion; and
c) recycling the dispersion to obtain a nano dispersion having appropriately sized nanotized particles.

21. The process as claimed in claim 17, wherein the component having particle size in the nano-scale range is the anti-microbial agent.

22. A method for treating a subject either suspected of having or having microbial infection, said method comprising act of administering to the subject an anti-microbial composition as claimed in claim 1.

23. The method as claimed in claim 22, wherein the microbial infection is selected from a group comprising fungal infection, bacterial infection and viral infection or any combination thereof; and wherein the anti-microbial agent is selected from a group comprising anti-fungal agent, anti-bacterial agent and anti-viral agent or any combination thereof.

24. The method as claimed in claim 23, wherein the fungal infection is caused by fungi selected from a group comprising Malassezia species, Trychophyton rubrum, Trychophyton mentagrophytes, Microsporum species, Epidermophyton species, Candida albicans and nondermatophyte molds or any combination thereof; wherein the bacterial infection is caused by bacteria selected from a group comprising Propionbacterium acnes, Staphylococcus species and Escherichia coli or any combination thereof; and wherein the viral infection is caused by virus selected from a group comprising herpes simplex virus, human cytomegalovirus, human adenovirus, hepatitis virus and human immunodeficiency virus or any combination thereof.

25. The method as claimed in claim 22, wherein the subject is a mammal including a human.

26. The method as claimed in claim 22, wherein the administering of the composition is by a route selected from a group comprising oral, topical, dermal, mucosal, buccal and gum or any combination thereof.

27. Anti-microbial composition as claimed in claim 1, for use in treating microbial infection.

28. A kit for treating microbial infection, said kit comprising components selected from a group comprising antimicrobial agent, oil, fatty acid or ester thereof having less than 11 carbon atoms and excipient or any combination thereof along with an instruction manual.
